SEQUENCE LISTING (38 sequences)

SEQ ID NO: 1 - RNA, 196 nt, Maize necrotic streak virus
ugaugugaggaacguggacugugaugugguggugcgguaccauggcuggucaccaugguaaugcguagggcaacacaguucauuaagacucacugaugauggcacuaggcacgguucacccccauccuucgggagggcuauagggggugaccggguuacaccaccggaagaccggaacauugccuuugggcagccc

SEQ ID NO: 2 - RNA, 122 nt, Maize necrotic streak virus
agauaucgaccugccugaccaggcugagauugcgcuagccggcguaguugguaucucucgcgcaagcggguuugaaggugcggccuaccuuagggggguaaauuguaacuucgcacaaaggc

SEQ ID NO: 3 - RNA, 32 nt, Maize necrotic streak virus
gaccaacaacucggcacacaaacgcacacaac

SEQ ID NO: 4 - RNA, 47 nt, Maize necrotic streak virus
gaacaagaccaguucauggaugcagaauacgagcaagucaguaggcc

SEQ ID NO: 5 - DNA, 11 bp, Artificial
  cDNA RACE products
atgaaagatat

SEQ ID NO: 6 - DNA, 12 bp, Artificial
  cDNA RACE product
ggggggagatat

SEQ ID NO: 7 - DNA, 22 bp, Artificial
  Synthetic oligonucleotide
cttcaatttcaattgggctgga

SEQ ID NO: 8 - DNA, 22 bp, Artificial
  Synthetic oligonucleotide
cctccagcccaattgaaattga

SEQ ID NO: 9 - DNA, 25 bp, Artificial
  Synthetic oligonucleotide
tgaggcatctcgatttctcggtgtc

SEQ ID NO: 10 - DNA, 24 bp, Artificial
  Synthetic oligonucleotide
agcattcggttgcgtcactctcgt

SEQ ID NO: 11 - DNA, 20 bp, Artificial
  Synthetic oligonucleotide
cctccttacgcatcttgaac

SEQ ID NO: 12 - DNA, 27 bp, Artificial
  Synthetic oligonucleotide
ggaattccaccagcgtttggcgaatag

SEQ ID NO: 13 - DNA, 48 bp, Artificial
  Synthetic oligonucleotide
gaacaattgtaatacgactcactatagagatatcgacctgcctgacca

SEQ ID NO: 14 - DNA, 46 bp, Artificial
  Synthetic oligonucleotide
gaacaattgtaatacgactcactatagatatcgacctgcctgacca

SEQ ID NO: 15 - DNA, 26 bp, Artificial
  Synthetic oligonucleotide
tcccgggctgcccaaaggcaatgttc

SEQ ID NO: 16 - DNA, 19 bp, Artificial
  Synthetic oligonucleotide
gggctgcccaaaggcaatg

SEQ ID NO: 17 - DNA, 37 bp, Artificial
  Synthetic oligonucleotide
gtaatacgactcactatagaccaacaactcggcacac

SEQ ID NO: 18 - DNA, 40 bp, Artificial
  Synthetic oligonucleotide
gtaatacgactcactatagaacaagaccagttcatggatg

SEQ ID NO: 19 - DNA, 30 bp, Artificial
  Synthetic oligonucleotide
gcggtaccgtggcgtttcactatggtaatg

SEQ ID NO: 20 - DNA, 20 bp, Artificial
  Synthetic oligonucleotide
ataccaactacgccggctag

SEQ ID NO: 21 - DNA, 21 bp, Artificial
  Synthetic oligonucleotide
tctgctgatcctgtcatttcc

SEQ ID NO: 22 - DNA, 20 bp, Artificial
  Synthetic oligonucleotide
tcgttccatggcctactgac

SEQ ID NO: 23 - RNA, 32 nt, Maize necrotic streak virus
ggaacacaacucggcacacaaacgcacacaac

SEQ ID NO: 24 - DNA, 20 bp, Artificial
  T7 RNA polymerase promoter
taatacgactcactataggg

SEQ ID NO: 25 - RNA, 98 nt, Maize necrotic streak virus
gcgguaguggaagaggucggaacugcccaccgccuugguuaugacggguuucugucauacuauagcggcgcgaaguugcguacuuauucgaaugccgu

SEQ ID NO: 26 - DNA, 23 bp, Artificial
  Synthetic oligonucleotide sequence
taatacgactcactatagatatc

SEQ ID NO: 27 - DNA, 24 bp, Artificial
  Synthetic oligonucleotide sequence
taatacgactcactataagatatc

SEQ ID NO: 28 - DNA, 24 bp, Artificial
  Synthetic oligonucleotide sequence
taatacgactcactataggatatc

SEQ ID NO: 29 - RNA, 35 nt, Maize necrotic streak virus
gaccaacaacucggcacacaaacgcacacaacaug

SEQ ID NO: 30 - RNA, 50 nt, Maize necrotic streak virus
gaacaagaccaguucauggaugcagaauacgagcaagucaguaggccaug

SEQ ID NO: 31 - RNA, 15 nt, Maize necrotic streak virus
auggcuggucaccau

SEQ ID NO: 32 - RNA, 15 nt, Artificial
  Mutated kissing loop
auggcuggucaccau

SEQ ID NO: 33 - RNA, 54 nt, Maize necrotic streak virus
gauaucgaccugccugaccaggcugagauugcgcuagccggcguaguugguauc

SEQ ID NO: 34 - RNA, 54 nt, Artificial
  mutant of T-shaped domain
gauaucgaccugcccgaccgggcugagauugcgcuagccggcguaguugguauc

SEQ ID NO: 35 - RNA, 54 nt, Artificial
  Mutated T-shaped domain
gauaucgaccugccugauuaggcugagauugcgcuagccggcguaguugguauc

SEQ ID NO: 36 - RNA, 54 nt, Artificial
  Mutated T-shaped domain
gauaucgaccugcccaaccgggcugagauugcgcuagccggcguaguugguauc

SEQ ID NO: 37 - RNA, 54 nt, Artificial
  Mutated T-shaped domain
gauaucgaccugccuaaccaggcugagauugcgcuagccggcguaguugguauc

SEQ ID NO: 38 - RNA, 199 nt, Maize necrotic streak virus
ugaugaugugaggaacguggacugugaugugguggugcgguaccauggcuggucaccaugguaaugcguagggcaacacaguucauuaagacucacugaugauggcacuaggcacgguucacccccauccuucgggagggcuauagggggugaccggguuacaccaccggaagaccggaacauugccuuugggcagccc

(19) United States
(12) Patent Application Publication
(10) Pub. No.: US 2008/0171361 A1
(43) Pub. Date: Jul. 17, 2008

(54) CAP-Independent Translational Enhancer for Protein Synthesis in Wheat Germ Extract and Transgenic Cereals

(75) Inventor: Kay Marie Scheets, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(73) Assignee: The Board of Regents for Oklahoma State University, Stillwater, OK (US)

(21) Appl. No.: 11/972,311

(22) Filed: Jan. 10, 2008

Related U.S. Application Data
(60) Provisional application No. 60/884,445, filed on Jan. 11, 2007.

(51) Int. Cl. C12N 15/33; C07H 21/02

(52) U.S. Cl. 435/69.1; 536/23.72; 536/23.1

(57) ABSTRACT

Novel cap-independent translational enhancers (CITEs) from monocot-infecting members of the virus family Tombusviridae (e.g. Maize necrotic streak virus) are provided. The CITEs can be used to produce uncapped mRNA that is efficiently translated into protein, for example, in wheat germ extract or in transgenic cereals or grasses.

### CROSS REFERENCE TO RELATED APPLICATION

This application claims the benefit of copending U.S. provisional application Ser. No. 60/884,445, filed 11 Jan. 2007, the complete contents of which are incorporated herein by reference.

### STATEMENT REGARDING FEDERALLY SPONSORED RESEARCH

This invention was made using funds from grants from the National Science Foundation having grant number 0440916. The United States government may have certain rights in this invention.

### SEQUENCE LISTING

This application includes as the Sequence Listing the complete contents of the accompanying text file “Sequence.txt”, created Jan. 9, 2008, containing 7,789 bytes, hereby incorporated by reference.

### DESCRIPTION

### BACKGROUND OF THE INVENTION

1. Field of the Invention

The invention generally relates to the enhancement of eukaryotic protein synthesis in vitro and in vivo. In particular, the invention provides novel cap-independent translational enhancers (CITEs) from monocot-infecting members of the virus family Tombusviridae, and satellite viruses they support. The CITEs can be used to produce uncapped mRNA that is efficiently translated into protein, for example, in wheat germ extract or in transgenic cereals and grasses.

2. Background of the Invention

The cell free in vitro production of eukaryotic recombinant proteins is of great utility in the field of biotechnology. The most frequently used systems for cell-free protein production are those based on wheat germ extract (WGE). Several types of cell-free eukaryotic protein expression kits that employ WGE are commercially available.

Unfortunately, the natural 5′ and 3′ untranslated regions (UTRs) of most eukaryotic and prokaryotic mRNAs do not contain sequences that allow for efficient translation of the mRNA in WGE. Instead, most mRNAs require the addition of a 5′ ^{m}7G-cap and a poly(A) tail, both of which are found on the majority of mature eukaryotic mRNAs. Meeting this requirement in a commercial WGE translation system adds extra inconvenience and cost since expensive cap-analogs must be used during mRNA transcription. In addition, cap-analogs are also known to inhibit mRNA translation, so coupled transcription/translation systems, designed to speed up and simplify in vitro protein synthesis, must use the much less efficient uncapped transcripts.

In contrast to eukaryotic and prokaryotic cells, some viruses do not require 5′ ^{m}7G-caps and a poly(A) tails on mRNA in order to permit efficient translation. Instead, the mRNA of these viruses contains 3′ and 5′ untranslated regions (UTRs) that act as cap-independent translation enhancers (CITEs). Base pairing between the two UTRs results in the development of secondary structural elements that promote efficient mRNA translation. A CITE from Barley yellow dwarf virus (BYDV) was identified (Guo, 2000; Guo, 2001; U.S. Pat. No. 5,910,628 to Miller et al., the entire contents of which is hereby incorporated by reference) and has been incorporated into a WGE kit for cell-free protein expression by Promega (Hurst et al, 2005). However, the efficiency of the BYDV CITE is low.

Other CITES are also known, e.g. Tobacco necrosis virus (TNV) has a BYDV-like CITE that is active in WGE (Meulewaeter et al, 2004; Shen and Miller, 2004) and its satellite (STNV) also has 3′ CITE activity that functions in WGE (Danthinne et al, 1993; Gazo et al, 2004; Timmer et al, 1993; van Lipzig et al, 2002). Other CITE activities have been reported in members of the Tombusviridae family (Koh et al, 2002; Mizumoto et al, 2003; Qu and Morris, 2000). However, none of the viruses in which these CITEs were identified naturally infect monocot plants, and it is unlikely that such CITEs would provide translational enhancement in transgenic monocot plants.

The prior art has thus-far failed to provide a practical, easily implemented and yet highly efficient tool for obviating the mRNA capping and poly-A tail requirements in cell-free protein translation systems and in transgenic cereals and grasses.

### SUMMARY OF THE INVENTION

The present invention is based in the discovery, identification and characterization of highly efficient, novel cap-independent translational enhancers (CITEs) from members of the virus family Tombusviridae, and satellite viruses supported by tombusvirids. The CITEs are from viruses that have the ability to naturally infect monocots (i.e. cereals and grasses). Therefore, the CITEs have the ability to function in hosts such as transgenic cereals and grasses. When used in the context of an in vitro protein translation system (such as a WGE system), the new CITEs permit economic synthesis of proteins without the requirement for expensive, translation-inhibiting cap-analogs. The CITEs may also be advantageously used for in vivo protein translation in recombinant, genetically engineered organisms. A prototypic, representative CITE has been isolated from Maize necrotic streak virus (MNeSV). The sequence of the exemplary MNeSV CITE (both genomic and subgenomic sequences) and descriptions of several uses of the new CITEs are provided herein. In particular, the use of the CITEs for in vitro expression of various proteins, polypeptides and peptides of interest, is described.

The invention provides a method of producing a protein, polypeptide or peptide translated from an uncapped eukaryotic messenger ribonucleic acid (mRNA). The method comprises the steps of: a) providing a DNA molecule comprising sequences encoding i) an RNA polymerase promoter; ii) a 5′ untranslated region (UTR) of a cap-independent translation enhancer (CITE) from a tombusvirid or a tombusvirid satellite virus, wherein said tombusvirid naturally infects monocots; iii) an open reading frame (ORF) encoding a protein; and iv) a 3′ UTR of a CITE from a tombusvirid or a tombusvirid satellite virus; b) allowing transcription of an uncapped mRNA molecule; and c) allowing translation of said uncapped mRNA molecule to produce said protein, polypeptide or peptide.

In one embodiment of the invention, the method is carried out in an in vitro translation system. In this case, the DNA molecule further comprises a restriction site immediately downstream of said 3′ UTR and the step of allowing transcription comprises contacting the DNA molecule with an RNA polymerase under conditions where the RNA polymerase binds to the RNA polymerase promoter and causes transcription of the sequences encoding the 5′ UTR of a CITE from a tombusvirid or a tombusvirid satellite virus, the ORF, and the 3′ UTR of a CITE from a tombusvirid or a tombusvirid satellite virus, into a uncapped mRNA molecule. Thus, in this embodiment, an RNA polymerase is provided to or with an in vitro translation mix. In one embodiment, the in vitro translation system is a wheat germ extract translation system.

In another embodiment of the invention, the method is carried out within a genetically engineered eukaryotic host cell such as a plant cell. In this embodiment, the DNA molecule further comprises sequences encoding a ribozyme and an RNA polymerase terminator sequence immediately downstream of the 3′ UTR. In one embodiment, the eukaryotic cell is a monocot. According to some embodiments of the invention, the tombusvirid is Maize necrotic streak virus. In some embodiments, the protein, polypeptide or peptide is a heterologous protein, polypeptide or peptide, examples of which include but are not limited to enzymes, fluorescent proteins, fusion (chimeric) proteins, structural proteins, transport proteins, regulatory proteins, and storage proteins.

The invention further provides a DNA molecule comprising sequences encoding an RNA polymerase promoter; a 5′ UTR of a CITE from a tombusvirid or a tombusvirid satellite virus, wherein the tombusvirid naturally infects monocots; an open reading frame (ORF) encoding a protein, polypeptide or peptide; and a 3′ UTR of a CITE from a tombusvirid or a tombusvirid satellite virus, wherein the tombusvirid naturally infects monocots. In one embodiment of the invention, the DNA molecule, further comprises a restriction site immediately downstream of the 3′ UTR. In some embodiments, the DNA molecule comprises sequences encoding a ribozyme and a RNA polymerase terminator sequence immediately downstream of the 3′ UTR. The protein, polypeptide or peptide that is encoded may be a heterologous protein, polypeptide or peptide, examples of which include but are not limited to enzymes, fluorescent proteins, fusion proteins, structural proteins, transport proteins, regulatory proteins, and storage proteins.

The invention further provides an isolated and substantially purified RNA molecule comprising the nucleic acid sequence:

(SEQ ID NO: 1)

UGAUGUGAGGAACGUGGACUGUGAUGUGGUGGUGCGGUACCAUGGCUGGU
CACCAUGGUAAUGCGUAGGGCAACACAGUUCAUUAAGACUCACUGAUGAU
GGCACUAGGCACGGUUCACCCCCAUCCUUCGGGAGGGCUAUAGGGGGUGA
CCGGGUUACACCACCGGAAGACCGGAACAUUGCCUUUGGGCAGCCC.

In another embodiment, the invention provides an isolated and substantially purified RNA molecule comprising the nucleic acid sequence:

(SEQ ID NO: 2)

AGAUAUCGACCUGCCUGACCAGGCUGAGAUUGCGCUAGCCGGCGUAGUUG
GUAUCUCUCGCGCAAGCGGGUUUGAAGGUGCGGCCUACCUUAGGGGGGUA
AAUUGUAACUUCGCACAAAGGC.

In yet another embodiment, the invention provides an isolated and substantially purified RNA molecule comprising the nucleic acid sequence:

| | |
|---|---|
| GACCAACAACUCGGCACACAAACGCACACAAC. | (SEQ ID NO: 3) |

In another yet another embodiment, the invention provides an isolated and substantially purified RNA molecule comprising the nucleic acid sequence:

(SEQ ID NO: 4)

GAACAAGACCAGUUCAUGGAUGCAGAAUACGAGCAAGUCAGUAGGCC.

The invention further provides an RNA molecule, comprising: a 5′ UTR of a CITE from a tombusvirid or a tombusvirid satellite virus, wherein said tombusvirid naturally infects monocots; a 3′ UTR of a CITE from a tombusvirid or a tombusvirid satellite virus, wherein the tombusvirid naturally infects monocots; and a heterologous set of nucleotides coding for a protein of interest. In one embodiment, the 5′ UTR is represented by SEQ ID NO: 2, SEQ ID NO: 3, or SEQ ID NO: 4, and the 3′ UTR is represented by SEQ ID NO: 1.

### BRIEF DESCRIPTION OF THE DRAWINGS

FIG. 1A and B. Structural components of MNeSV CITE on genomic RNA (gRNA). A, the 5′ UTR (SEQ ID NO: 2) and B, the 3′UTR (SEQ ID NO: 1) of genomic RNA (gRNA) are depicted. TSD=T-shaped domain; KL=“kissing loop”.

FIG. 2A and B. Schematic representation of cDNAs with 3′ UTR and 5′ UTR of MNeSV genomic or subgenomic RNAs. Transcription produces mRNA with base-pairing interactions and structures that promote cap-independent translation to express a protein encoded by the open reading frame (ORF). The asterisk represents a 5′ ^{m}7G cap. A) in vitro translation; B) in vivo translation.

FIG. 3. Primer extension mapping of the 5′ termini of the MNeSV viral RNA (vRNA) and subgenomic mRNA (sgRNA). Primers (see Materials and Methods for sequences) used for each reaction are listed at the top of the lanes. RNA from two different virion preparations was used for extension with the 5′RO primer. The products were run next to a sequencing ladder for a 5′ RLM-RACE cDNA using the same primer. To identify the 5′ ends of the viral sgRNAs, total RNA from infected (+) or healthy (−) plants and t8-6 RNA (Tr) were used as templates, and the sequencing ladder was generated using p8-6. Arrowheads mark the sgRNA 5′ ends, and all sequences represent (+) RNAs.

FIG. 4A-C. Predicted RNA folding structures for the 5′ UTR, 3′UTR and putative internal replication element of MNeSV. A) 5′UTR structure (SEQ ID NO: 2) that most closely resembles the regions identified in TBSV (Ray et al., 2003), predicted as fourth lowest energy folding by mfold version 3.1 (Zuker, 2003). Sequences that are identical in all tombusviruses are italicized. The T-shaped domain (TSD) and downstream domain (DSD) are marked with dashed lines. The central stem loop (SL5) corresponds to the fifth stem loop in TBSV. The intervening sequence (is5/6), bulge 1 (B1) and bulge 2 (B2) regions of the DSD are marked. Bases in open circles may base pair like the pseudoknot in TBSV, and bases complementary to the loop in the MNeSV 3′UTR are white letters in black circles. B) The single structure predicted by mfold version 3.1 for nt 1227-1324 (SEQ ID NO: 25), the putative internal replication enhancer. The large loop (L3) with a CC mismatch (boxed Cs) has potential Watson/Crick (dashed) or non-Watson/Crick (dotted) base pairs marked, and the bulge (B2) and stem (S2A) correspond to regions in the alternative structure of the region II hairpin of TBSV DI-RNA (Monkewich et al., 2005). C) The lowest energy 3′UTR structure (SEQ ID NO: 38) predicted by mfold version 2.3 at 37° C. and 28° C. The p21 stop codon is italicized. An arrow marks the 3′ end of Asp718 I-truncated transcripts. Italicized sequences are identical to sequences in CBLV, and lower case letters show differences for the CBLV sequence. The asterisks represent one nt deletions in CBLV. Bases complementary to the first TSD loop in the MNeSV 5′UTR are white letters in black circles, while gray circles mark bases in the putative replication silencer element (RSE) and its complement at the 3′ end (Fabian et al., 2003). A rectangle encloses the region with high sequence identity to other tombusviruses.

FIG. 5A-C. Comparison of genome maps of MNeSV and TBSV and construction of full-length MNeSV cDNAs. A) Open boxes represent major ORFs, and the leaky stop codons (UGA) found in the RdRp ORFs are indicated. sgRNA start sites (sg1 and sg2) are marked by bent arrows below the genomes. The MNeSV nonhomologous CP ORF is represented as a gray box. The locations of regulatory elements are marked by arrows. Long distance base-pairing motifs affecting transcription have connected arrows above the genomes while long distance base-pairing motifs affecting translation have connected arrows below the genomes. Replication regulators include the T-shaped domain, stem loop 5, downstream domain (TSD, SL5, DSD) (Ray et al., 2003; Wu et al., 2001), internal replication element RII (IRE RII) (Monkewich et al., 2005) and replication silencing element (RSE) (Fabian et al., 2003; Pogany et al., 2003). sgRNA regulatory elements include activator sequence-1, stem loop 1sg1, receptor sequence-1 (AS 1, SL1sg1, RS1) (Choi and White, 2002), AS2, RS2 (Lin and White, 2004), distal element and core element (DE, CE) (Zhang et al., 1999). Cap-independent translational enhancer (CITE) (Fabian and White, 2004; Wu and White, 1999). The TBSV figure is modified from Lin and White (2004). B) The four cDNA fragments used to construct full-length MNeSV cDNAs are indicated with heavy lines. The ssDNA overlap regions of MNeSV2.1 and MNeSV2.2 are shown with thin lines. The locations of restriction sites used for cDNA construction and transcription template linearizations are marked. C) The sequences at the 5′ ends of the transcription template cDNAs are shown next to the T7 RNA promoter sequence which is italicized (SEQ ID NOS: 26-28).

FIG. 6A-C. Infectivity of in vitro synthesized MNeSV transcripts and vRNA in protoplasts and plants. BMS protoplasts were inoculated with vRNA, uncapped (−), or capped (+) transcripts from three cDNA clones or mock-inoculated (none), and samples for total RNA and CP were collected at 48 hours post-inoculation for analysis. A) Phosphorimage of a northern blot to detect (+) RNA with a 3′ end probe. Locations of genomic RNA (gRNA) and sgRNAs are marked. The ethidium bromide-stained 18S rRNA below the phosphorimage shows loading levels. B) CP accumulation in protoplasts assayed by PAS-ELISA. C) Maize seeds were inoculated by VPI with uncapped or capped transcripts or infected plant extract (far right), and seedlings were monitored for infection. Not all seeds germinated after VPI, so the fraction represents number infected/number germinated and is the sum of two independent experiments.

FIG. 7. Complementarity of sequences in the MNeSV sgRNA 5′ ends and the 3′ UTR kissing loop. The 5′ ends of sgRNA1 (SEQ ID NO: 29) and sgRNA2 (SEQ ID NO: 30) including the UTRs and start codons for CP, p21, and p19 are aligned with the 15 nt sequence containing the kissing loop (3′KL, SEQ ID NO: 31). The start codons are indicated in larger bold font. Complementary sequences are bold, and bases that are paired in the stem in FIG. 4C are underlined. The same alignment is shown for the transcript from pNsg2-KLC (3′KLC, SEQ ID NO: 32), and mutated bases are shown in lower case. The underlined regions containing mutations in pNsg2-KLC are predicted to base pair in the stem the same as wild type.

FIG. 8. In vitro translation products of MNeSV vRNA and uncapped gRNA or sgRNA transcripts from wheat germ extracts. Water (none) or 0.5 pmol of vRNA (vRNA), full-length transcripts of gRNA (2-2) or sgRNAs (sgRNA1 and sgRNA2) were used. For one translation, t2-2 and the two sgRNA transcripts (3 in 1) were mixed before adding the RNAs to the WGE. Equal volumes of translation reactions were separated by SDS-PAGE, electroblotted to nitrocellulose, and exposed to a phosphorimager. Sizes of molecular weight markers (MW) are on the right, and the location of p89 is marked with an arrow.

FIG. 9. Effects of ^{m}7GpppG caps and 3′ truncations on translation of MNeSV gRNA transcripts in wheat germ extracts. Water (none) or 0.5 pmol of vRNA, uncapped (−) or capped (+) full length transcripts (2-2) or transcripts missing the 3′ 156 nt (2-2Δ3′) were used. Sizes of molecular weight markers (MW) are on the right and the proteins were analyzed as in FIG. 8. The levels of p30 produced relative to the uncapped full length t2-2 reaction are shown below the lanes and represent the mean±standard error for four experiments using the phosphorimager for quantitation. Triangles mark the locations of CP and p21, and asterisks mark proteins synthesized from internal methionines in ORFs 1 and 2.

FIG. 10A and B. Effects of ^{m}7GpppG caps and 3′ truncations on translation of sgRNA transcripts. Water (none) or 0.5 pmol of uncapped (−) t2-2 (2-2), uncapped and capped (+) full-length wild type (wt) tNsg1 and tNsg2, and tNsg2-KLC (sg2-KLC), or transcripts missing the 3′ 156 nt (Δ3′) were used to program WGE. For one reaction, equimolar amounts of uncapped t2-2, tNsg1, and tNsg2 were mixed, and 1.5 pmol of the mixture was translated by WGE (3 in 1). Sizes of molecular weight markers (MW) are on the right and proteins were analyzed as in FIG. 8. A) sgRNA1 transcripts. The levels of CP relative to uncapped tNsg1 are given below the lanes and represent the mean±standard errors of four experiments. B) sgRNA2 transcripts. The levels of p21 (black triangles) and p19 (open triangles) relative to uncapped tNsg2 are shown and represent the mean±standard deviation of three experiments for wt and Δ3′ and two experiments for tNsg2-KLC.

FIG. 11A-C. Effects of mutating bases in the kissing loop of the 5′ UTR of genomic RNA transcripts on translations in wheat germ extract. A) T-shaped domains of wild type (2-2, SEQ ID NO: 33) and mutant transcripts (TSDm1, SEQ ID NO: 34; TSDm2, SEQ ID NO: 35; TSD13, SEQ ID NO: 36; TSDm3, SEQ ID NO: 37). Mutated bases are lower case. B) Genomic RNA map indicating the p30 and p89 ORFs. An arrow indicates the location of the mutations, and the asterisk marks the location of the suppressible stop codon. C) Water (none) or 0.5 pmol of uncapped full length transcripts were used. Sizes of molecular weight markers (MW) are on the right and the proteins were analyzed as in FIG. 8. The levels of p30 produced relative to the full length 2-2 reaction are shown below the lanes using the phosphorimager for quantitation. Solid triangle marks the location of p89 and an open triangle marks the location of p30.

FIG. 12A and B. Effects of moving the 3′ UTR of genomic RNA transcripts closer to the 5′ UTR on translations in wheat germ extract. A) Maps of full length (2-2) and shortened genomic transcripts indicating the restriction sites used for deletion of interior sequences between the p30 ORF and the 3′ UTR. The gray box indicates a polylinker region, the asterisk marks the location of the suppressible stop codon, and the sizes of the transcripts are indicated on the right. B) Water (none) or 0.5 pmol of uncapped transcripts were used. Sizes of molecular weight markers (MW) are on the left and the proteins were analyzed as in FIG. 8. The levels of p30 produced relative to the full length 2-2 reaction are shown below the lanes using the phosphorimager for quantitation. Solid triangle marks the location of p89 and an open triangle marks the location of p30.

### DETAILED DESCRIPTION OF THE PREFERRED EMBODIMENTS OF THE INVENTION

Novel, highly efficient cap-independent translational enhancers (CITEs) from monocot-infecting members of the virus family Tombusviridae, as well as satellite viruses supported by tombusvirids, are provided by the present invention. The viruses from which the CITEs originate are capable of naturally infecting cereals and grasses, and thus the CITEs have the advantageous ability to function in transgenic cereal and grass hosts to increase production of a protein of interest. The CITEs comprise 3′ and 5′ untranslated regions (UTRs) of the viral genome, as well as the subgenomic equivalents of those genomic sequences. By “subgenomic equivalent” we mean: the 5′ and 3′ UTRs of a viral sequence generated during a viral infection that produce a mRNA less than the full length of the genomic RNA and is coterminal with the 3′ end of the genomic RNA. When present at the 3′ and 5′ ends of an mRNA transcript containing an open reading frame (ORF) that encodes a protein, the CITEs permit translation of the mRNA without capping. Transcription-translation coupling efficiency is thus high in systems using the CITEs, since cap-analogs, which inhibit translation, need not be present. The CITEs of the invention are ideal candidates for use in protein translation systems, either in vitro (e.g. WGE systems) or in vivo (e.g. in genetically engineered organisms).

In one exemplary embodiment, the CITE is isolated from Maize necrotic streak virus (MNeSV) and comprises the 3′ untranslated region (UTR) of MNeSV (nucleotides 3899-4094) having the following sequence:

(SEQ ID NO: 1)

UGAUGUGAGGAACGUGGACUGUGAUGUGGUGGUGCGGUACCAUGGCUGGU
CACCAUGGUAAUGCGUAGGGCAACACAGUUCAUUAAGACUCACUGAUGAU
GGCACUAGGCACGGUUCACCCCCAUCCUUCGGGAGGGCUAUAGGGGGUGA
CCGGGUUACACCACCGGAAGACCGGAACAUUGCCUUUGGGCAGCCC,
and a 5′ UTR of MNeSV having one of the following
ribonucleotide sequences: (nucleotides nt 1-122)

(SEQ ID NO: 2)

AGAUAUCGACCUGCCUGACCAGGCUGAGAUUGCGCUAGCCGGCGUAGUUG
GUAUCUCUCGCGCAAGCGGGUUUGAAGGUGCGGCCUACCUUAGGGGGGUA
AAUUGUAACUUCGCACAAAGGC;
(nucleotides 2488-2419)

(SEQ ID NO: 3)

GACCAACAACUCGGCACACAAACGCACACAAC;
and
(nucleotides 3314-3360)

(SEQ ID NO: 4)

GAACAAGACCAGUUCAUGGAUGCAGAAUACGAGCAAGUCAGUAGGCC.

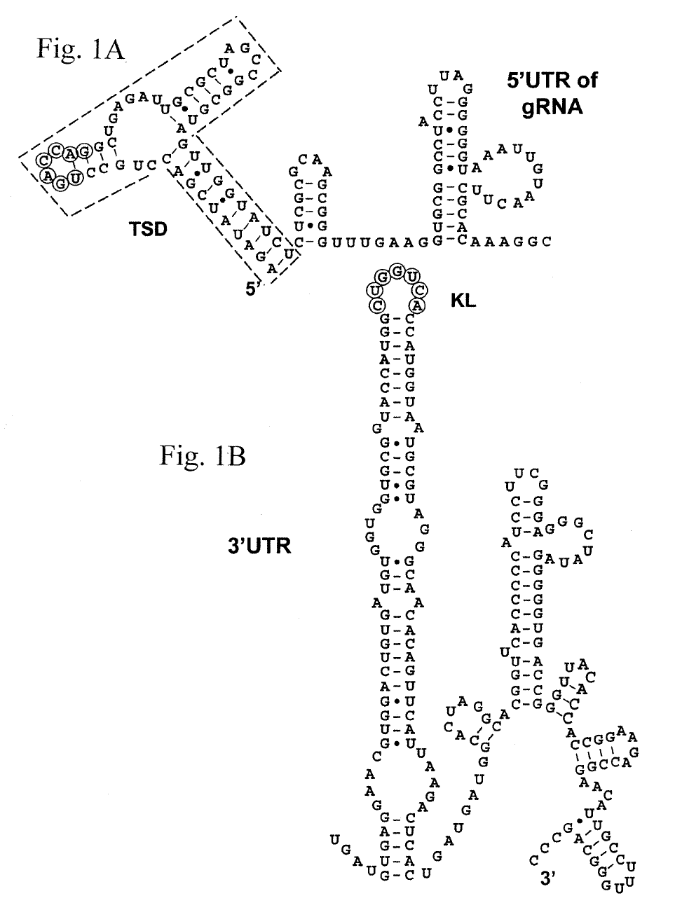

The UTRs of genomic RNA are represented in FIG. 1, which shows the probable secondary structure of both regions and the bases capable of pairing between them. Other monocot-infecting members of the family Tombusviridae such as Maize chlorotic mottle virus, Panicum mosaic virus which supports a satellite virus (SPMV), Johnson grass chlorotic streak mosaic virus, Oat chlorotic streak virus, Maize white line mosaic virus which supports a satellite virus (SMWLMV), and Maize mild mottle virus are likely to contain structures and sequences in their 5′ and 3′ UTRs that provide CITE activity similar to that of MNeSV. Such sequences may also be used in the practice of the invention.

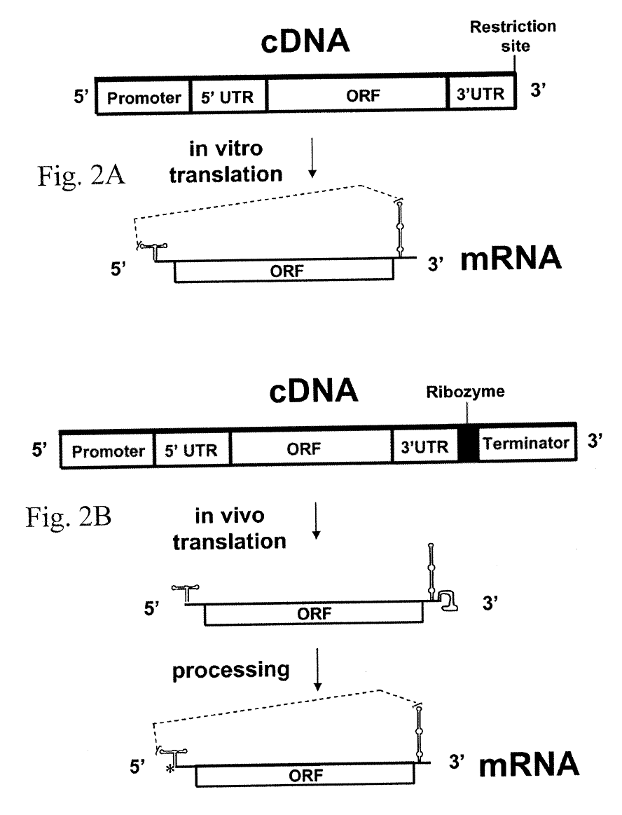

The CITEs of the invention may be utilized to carry out the production of protein (i.e. protein translation) from uncapped mRNA. This may occur, for example, in an in vitro translation system such as a WGE system. For this purpose, correct positioning of the 3′ and 5′ CITE sequences is required. With reference to FIG. 2A, this is generally accomplished by making a cDNA construct containing an RNA polymerase promoter operably linked to the cDNA sequence encoding a 5′ UTR of either genomic RNA or subgenomic RNA. This sequence is followed by cDNA containing an open reading frame (ORF), including the start and stop codons, encoding the protein to be expressed. The cDNA sequence representing the 3′ UTR of the CITE is located downstream of the protein ORF. In addition, for cDNAs constructed for in vitro synthesis of mRNAs, a restriction site unique to the region is located at the 3′ end of the 3′ UTR, allowing the cDNA to be linearized and permitting correct termination of the mRNA. With this construct, uncapped RNA that initiates at the beginning of the 5′ UTR and terminates at the end of the 3′ UTR can be transcribed in vitro. The in vitro synthesized mRNA made by this process can then be used to program WGE to synthesize large amounts of the protein without using a 5^{m}7G-cap analog or poly(A) tail. Such in vitro protein expression may be carried out, for example, using in vitro expression systems such as those which utilize WGE.

Those of skill in the art will recognize that the extreme 5′ end of the UTRs to be used in an in vitro system may be modified slightly so large amounts of RNA can be readily synthesized. As an example, if T7 RNA polymerase will be used to synthesize the RNA, then the first three nucleotides of the transcript may be GGG since these three bases terminate the T7 RNA polymerase promoter (TAATACGACTCACTATAGGG [SEQ ID NO: 24]) and T7 RNA polymerase initiates synthesis at the second G of the promoter sequence. Thus the RNA transcript produced from an expression plasmid based on the genomic 5′ UTR may start with GGGAUAUC instead of AGAUAUC by adding one extra G at the 5′ end and replacing the initial A with a second G. Similar changes may be advantageous for expression vectors based on the 5′ UTRs of sgRNA1 or sgRNA2. Those of skill in the art will recognize that analogous changes may also be advantageous when other promoters (such as the T3 RNA polymerase promoter or the SP6 RNA polymerase promoter, etc.) are employed, in order to optimize RNA synthesis from the promoter.

For in vivo-expressed RNA, a similar construct is utilized. With reference to FIG. 2B, the positioning of the promoter, 5′ UTR, ORF and 3′UTR are essentially the same. However, the 3′ end of the mRNA is correctly terminated by encoding DNA for a ribozyme and an RNA polymerase terminator sequence following the 3′ UTR. From this cDNA, RNA can be transcribed in vivo by nuclear RNA polymerases and it may be capped by nuclear capping enzymes. The ribozyme cleaves the mRNA immediately downstream of the 3′ UTR to produce mature mRNA. Such in vivo protein expression may be carried out, for example, in plants that have been genetically engineered to contain DNA that encodes the elements of the construct as described above, e.g. cereals such as maize (Zea mays), wheat (Triticum aestivum), barley (Hordeum vulgare), rice (Oryza sativa), sorghum (Sorghum bicolor), and grasses such as sugar cane (Saccharum officinarum), and switchgrass (Panicum virgatum).

In some embodiments of the invention, the sequences of the 3′ and 5′ UTRs that make up the CITEs described herein are represented, for example, by SEQ ID NOS: 1 and 2, as well as corresponding subgenomic sequences, and corresponding sequences from other monocot-infecting members of the family Tombusviridae. However, those of skill in the art will recognize that several possible variations of these sequences may also be utilized in the practice of the invention. For example, certain nucleotide substitutions, additions, or deletions may be tolerated without changing the ability of the CITE to promote translation from uncapped mRNA. In general, the primary sequence of the UTRs may be altered in any manner, as long as the ability to maintain long distance base-pairing between the 5′ and 3′ UTRs illustrated in the structures depicted in FIG. 1 is preserved. Such sequences may generally be at least about 50%, or 60%, or preferably about 70%, or more preferably about 80%, and most preferably about 90% or more identical to the sequences represented by SEQ ID NOS: 1, 2, 3 and 4.

In an embodiment of the invention, the CITEs described herein are used to express various proteins, polypeptides, or peptides of interest in in vitro translation systems. Such proteins, polypeptides, or peptides may be viral, prokaryotic or eukaryotic in origin, and may include recombinant, mutant and chimeric forms thereof. Generally, the proteins, polypeptides or peptides are heterologous, i.e. they do not originate from Maize necrotic streak virus, although this need not be the case. Exemplary proteins, polypeptides, and peptides include but are not limited to enzymes such as firefly luciferase, fluorescent proteins such as green fluorescent protein (GFP) from jellyfish and GFP-fusion proteins, structural proteins such as poliovirus coat protein, transport proteins such as potassium ion channel, regulatory proteins such as zinc finger transcription regulators, and storage proteins such as gliadin from wheat. Any protein, polypeptide, or peptide capable of being expressed in an in vitro translation system may be expressed using the CITEs of the present invention.

Accordingly, the invention also provides constructs for in vitro expression of such proteins, polypeptides, and peptides. Such constructs include RNA encoding the protein, polypeptide, or peptide of interest, flanked by suitable CITE elements as described herein.

### EXAMPLES

### Example 1

### Infectious cDNA Transcripts of Maize Necrotic Streak Virus: Infectivity and Translational Characteristics.

Maize necrotic streak virus (MNeSV) is a unique member of the family Tombusviridae that is not infectious by leaf rub-inoculation and has a coat protein lacking the protruding domain of aureusviruses, carmoviruses, and tombusviruses (Louie et al., Plant Dis. 84, 1133-1139, 2000). Completion of the MNeSV sequence indicated a genome of 4094 nt. RNA blot and primer extension analysis identified subgenomic RNAs of 1607 and 781 nt. RNA and protein sequence comparisons and RNA secondary structure predictions support the classification of MNeSV as the first monocot-infecting tombusvirus, the smallest tombusvirus yet reported. Uncapped transcripts from cDNAs were infectious in maize (Zea mays L.) protoplasts and plants. Translation of genomic and subgenomic RNA transcripts in wheat germ extracts indicated that MNeSV has a 3′ cap-independent translational enhancer (3′CITE) located within the 3′ 156 nt. The sequence, predicted structure, and the ability to function in vitro differentiate the MNeSV 3′CITE from that of Tomato bushy stunt virus.

### Introduction

Maize necrotic streak virus (MNeSV) is a monopartite single-stranded positive sense RNA virus, and partial genome sequence analysis showed it to be related to members of the family Tombusviridae (Louie et al., 2000). The genome organization of MNeSV was found to be similar to that of tombusviruses and aureusviruses with five open reading frames (ORFs). Nonstructural proteins encoded by MNeSV were most similar to tombusviruses even though the estimated size (˜4.3 kb) of virion RNA (vRNA) was closer to that of aureusviruses. Analysis of the predicted 27.4 kDa coat protein (CP) of MNeSV indicated that it did not contain the protruding domain found on tombusvirus and aureusvirus CPs and was most closely related to CPs of necroviruses. The virion size of 32 nm was closer to that of tombusviruses (32-35 nm) (Lommel et al., 2005b) than necroviruses (28 nm) (Lommel et al., 2005a). Similarly to tombusviruses, no insect vector of MNeSV was identified among eight insect species tested (Louie et al., 2000). A characteristic of MNeSV that differentiated it from previously identified tombusviruses was that the virus could not be transmitted by leaf rub inoculation (Louie et al., 2000). However, MNeSV readily infected maize (Zea mays L.) via vascular puncture inoculation (VPI) of seeds.

Several species of dicot-infecting tombusviruses have been used to study various aspects of the virus life cycle: replication in vivo (Fabian et al., 2003; Ray et al., 2003; Wu et al., 2001), in vitro (Nagy and Pogany, 2000; Panavas et al., 2002) and in yeast (Panavas and Nagy, 2003; Pantaleo et al., 2003); recombination and production of defective interfering-RNAs (DI-RNAs) (Burgyan et al., 1991; Reade et al., 1999; Scholthof, K. et al., 1995); transcription (Choi et al., 2001; Choi and White, 2002; Lin and White, 2004); in vivo translation (Fabian and White, 2004; Wu and White, 1999); cell-to-cell and long-distance movement (Chu et al., 2000; Scholthof, H. et al., 1995); initiation and suppression of gene silencing (Havelda et al., 2003; Qiu et al., 2002); and fungal transmission (McLean et al., 1994). The similarity of MNeSV to tombusviruses suggested that it might be the first monocot-infecting tombusvirus, and could thus provide a very useful tool to compare aspects of the tombusviral life cycle in a monocot system.

The genomes of viruses in the family Tombusviridae are uncapped and do not contain poly(A) tails, two structures required for efficient translation of most eukaryotic mRNAs. For plant mRNAs, the m7GpppN cap is bound by initiation factor eIF4F or eIFiso4F, the poly(A) tail is coated by poly(A) binding protein, and both proteins bind to additional initiation factors to form a circular structure which efficiently recruits the 40S ribosome subunit (Kawaguchi and Bailey-Serres, 2002). Many viruses use alternative 5′ and/or 3′ structures to efficiently translate their mRNAs. In the family Tombusviridae, 3′ translational enhancers (3′TEs) have been previously identified in Tomato bushy stunt virus-C (TBSV-C) (Wu and White, 1999), the necroviruses Tobacco necrosis virus-A (TNV-A) and TNV-D (Meulewaeter et al., 2004; Shen and Miller, 2004) and their satellite virus STNV (Danthinne et al., 1993; Timmer et al., 1993), the dianthovirus Red clover necrotic mosaic virus (Mizumoto et al., 2003), and the carmoviruses Turnip crinkle virus (Qu and Morris, 2000) and Hibiscus chlorotic ringspot virus (Koh et al., 2002). TBSV does not naturally infect cereals, and its 3′ cap independent translational enhancer (3′ CITE) is active in cucumber protoplasts but not in wheat germ extract (WGE) (Wu and White, 1999). As a monocot-infecting tombusvirus, we hypothesized that MNeSV might provide a tool for analyzing cap independent translation of a tombusvirus in WGE, a system used for the well-studied 3′TE of Barley yellow dwarf virus-PAV (BYDV-PAV) (Allen et al., 1999) and other viruses.

In this Example, we report the completed sequence of MNeSV and the construction of infectious transcript cDNAs. We also mapped the two subgenomic RNAs (sgRNAs) and identified predicted secondary structures in MNeSV genomic RNA (gRNA) similar to those found for dicot tombusviruses. Lastly, we have identified a cap independent translational enhancer (CITE) in the 3′ untranslated region (UTR) of MNeSV that functions on gRNA and sgRNAs in WGE. These results indicate that MNeSV should be classified as a tombusvirus.

### Materials and Methods

Cloning and sequencing 5′ and 3′ ends of MNeSV vRNA. The 5′ end of the vRNA was cloned using the First Choice RLM-RACE kit (Ambion, Austin, Tex.) except that reverse transcription was performed with Thermoscript (Invitrogen, Carlsbad, Calif.) using MNeSV primer 5′RLM-RACE (Table 1) according to the manufacturer's instructions. vRNA was either directly ligated to the RNA oligomer (5′ RACE Adapter) from the RLM-RACE kit or was pretreated with CIP and TAP before RNA ligation. The cDNA was amplified with the 5′ RACE Inner Primer and the phosphorylated primer #106255 using Vent DNA polymerase (New England Biolabs, Boston, Mass.) according to the manufacturer's recommendations. After BamHI digestion to cleave the 5′ RACE Inner Primer site, the fragment was gel purified and cloned into the SmaI and BamHI sites of Bluescript KS+ (Stratagene, La Jolla, Calif.). 5′ RACE (Invitrogen) was carried out according to the manufacturer's instructions using primer MNeSV2.1.1R for first strand cDNA synthesis and MNESV2 for amplification of the C-tailed cDNA. cDNAs were cloned into pGEM-4z (Promega, Madison, Wis.) for sequence analysis.

TABLE 1

Unique oligonucleotides used in cDNA constructions (SEQ IDs 7-22)

| | | | | |
|---|---|---|---|---|
| | | | | SEQ ID |
| Name | Sequence^{a} | Sense^{b} | Location^{c} | NO: |

| | | | | |
|---|---|---|---|---|
| #785 | cttcaatttcaattgggctgg(ddA)^{d} | | | 7 |
| #925 | cctccagcccaattgaaattga | | | 8 |
| 3′dsRNA | TGAGGCATCTCGATTTCTCGGT | + | 3651-3675 | 9 |
| | GTC | | | |
| 5′RLM-RACE | AGCATTCGGTTGCGTCACTCTC | − | 559-582 | 10 |
| | GT | | | |
| MNeSV2.1.1R | CCTCCTTACGCATCTTGAAC | − | 521-540 | 11 |
| MNESV2 | ggaattccACCAGCGTTTGGCGAAT | − | 417-435 | 12 |
| | AG | | | |
| MNeSV T7-2 | gaacaattgtaatacgactcactata(g/A)GAT | + | 1-21 | 13 |
| | ATCGACCTGCCTGACCA | | | |
| MNeSV T7-1 | gaacaattgtaatacgactcactataGATATC | + | 2-21 | 14 |
| | GACCTGCCTGACCA | | | |
| MNeSV | tcccGGGCTGCCCAAAGGCAATG | − | 4073-4094 | 15 |
| 3′SmaI | TTC | | | |
| MNe4076c | GGGCTGCCCAAAGGCAATG | − | 4076-4094 | 16 |
| T7/MNesg1 | gtaatacgactcactataGACCAACAACT | + | 2488-2506 | 17 |
| | CGGCACAC | | | |
| T7/MNesg2 | gtaatacgactcactataGAACAAGACCA | + | 3314-3335 | 18 |
| | GTTCATGGATG | | | |
| KLC | GCGGTACCgTGGCgttTCACtATG | + | 3932-3962 | 19 |
| | GTAATG | | | |
| 5′RO | ATACCAACTACGCCGGCTAG | − | 35-54 | 20 |
| sg1RO | TCTGCTGATCCTGTCATTTCC | − | 2535-2554 | 21 |
| sg2RO | TCGTTCCATGGCCTACTGAC | − | 3350-3369 | 22 |

^{aPK Upper case letters represent MNeSV cDNA sequence, and lower case letters are non-MNeSV. Restriction sites are italicized, and the T7 RNA polymerase promoter is underlined.}
b(+) viral strand, (−) complementary strand
^{c}Numbering refers to (+) strand of Genbank sequence.
^{d}dideoxyadenosine

The 3′ end was cloned using an anchored cDNA method (Weng and Xiong, 1995). T4 RNA ligase was used to ligate the phosphorylated DNA oligomer #785, which contains a MunI site, to the 3′ end of the MNeSV vRNA and reverse transcribed with Thermoscript using the complementary primer #925. PCR amplification using primers #925 and 3′dsRNA, which binds upstream of the SalI site in MNeSV, was followed by digestion with SalI and MunI, gel purification, and cloning into Bluescript SK+ digested with EcoRI and SalI. These plasmids were named p3′-1, p3′-2 and p3′-3. Both strands of 5′ and 3′ cDNA plasmid inserts were sequenced.

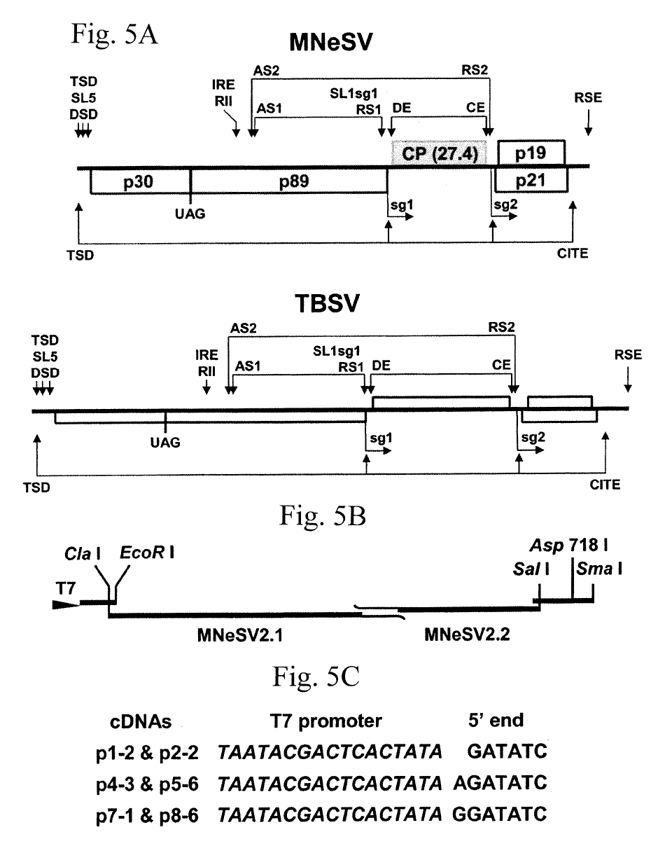

Construction of infectious cDNA transcript plasmids. Six different full-length clones were made using standard cloning procedures (Sambrook et al., 1989). The 5′ end of MNeSV was placed downstream of a T7 RNA polymerase promoter by PCR amplification from a 5′ RLM-RACE cDNA using the primers MNeSV T7-1 or MNeSV T7-2 and 5′RLM-RACE. The DNA was digested with MfeI and EcoRI, gel purified and ligated into EcoRI digested pUC119. Clones (pT71A, pT722, and pT725) were selected that carried GATATC, GGATATC or AGATATC, respectively, immediately following the T7 promoter and with the remaining polylinker region 3′ of the insert. A unique SmaI site was placed at the 3′ end by amplifying DNA from p3′-3 and p3′-1 using the M13 forward sequencing primer and phosphorylated MNeSV 3′SmaI. After digestion with SalI the gel-purified fragments were cloned into pT71A, pT722, and pT725 that each contained a Klenow-filled HindIII end and SalI sticky-end. Six different cDNAs with different 5′/3′ end combinations were digested with ClaI and SalI for further insertions. Plasmids MNeSV2.1 and MNeSV2.2 (Louie et al., 2000) contain 2.5 and 1.67 kb inserts, respectively, that overlap by 248 bp. Both plasmids were linearized in the polylinker region with XbaI, then digested with high concentrations of exonuclease III for 1 and 1.5 min at 37° C. to remove 250-500 nt of complementary strands, followed by phenol/CHCl3 extraction and precipitation (Schubert et al., 1988). MNeSV2.1 exonuclease III products were digested with ClaI, the MNeSV2.2 exonuclease III products were digested with SalI, and the inserts consisting of dsDNA with long 3′ ssDNA tails were gel purified, pooled and precipitated. The fragments were dissolved in 50 mM NaCl, briefly heated and allowed to anneal before ligating into the ClaI/SalI-prepared 5′/3′ plasmids (FIG. 5). The 5′ and 3′ ends and 500-800 bases at the overlap junctions were sequenced for all clones, and the p8-6 and p2-2 inserts were sequenced completely.

Construction of sgRNA transcription plasmids. The 5′ ends of sgRNA1 and sgRNA2 were placed downstream of a T7 RNA polymerase promoter using phosphorylated oligos T7/MNesg1 or T7/MNesg2 and MNe4076c to amplify regions of p2-2 using Vent DNA polymerase, and the fragments were ligated to SmaI digested pUC18. The resulting clones were named pNsg1 and pNsg2. To produce pNsg2-KLC, the M13 Reverse Sequencing Primer (-48) (New England Biolabs) and KLC were used to amplify the 3′ end of pNsg2 using Vent polymerase. After digestion with KpnI and gel purification, the 160 bp fragment was ligated into the large fragment of pNsg2 which was digested with KpnI and dephosphorylated. All sgRNA cDNA inserts were completely sequenced.

Primer extension analysis. The 5′ ends of sgRNAs were mapped by primer extension with Thermoscript reverse transcriptase (Scheets, 2000; Wang and Simon, 1997). Total RNA (3 μg) from healthy or MNeSV-infected maize plants or 0.5 μg of t8-6 (see below for synthesis) was annealed to 1 pmol of ³³P-labeled oligonucleotide sg1RO or sg2RO and reacted as described using 11 units of Thermoscript. One twelfth of each primer extension reaction was separated on an 8% polyacrylamide, 8 M urea sequencing gel along with dideoxy-termination sequencing reactions (Sequenase kit, Amersham, Piscataway, N.J.) of p8-6 using the same radiolabeled primers. Primer extension reactions of vRNA contained 0.2 μg vRNA and radiolabeled 5′RO with a sequencing ladder generated from a 5′ RLM-RACE plasmid. The dried gels were exposed to a phosphorimager (BioRad, Hercules, Calif.).

Protoplast inoculations and analysis. Capped transcripts were synthesized using the mMessage mMachine kit (Ambion) and uncapped transcripts were synthesized as in Scheets (2000). Protoplast inoculations were performed as in Scheets (2000). Briefly, BMS suspension culture protoplasts (1-1.5×10⁶) were inoculated with 10 μg of transcripts or vRNA using polyethylene glycol, and washed protoplasts were incubated in growth media with sampling at 0, 24, and 48 hours. Samples for RNA and CP quantitation were prepared and analyzed by northern blotting of agarose gels and PAS-ELISA, respectively, (Scheets, 2000) except that RNA probes were synthesized from p3′-1 linearized with SalI, and rabbit antiserum to MNeSV (Louie et al., 2000) was used in the PAS-ELISA.

Maize infectivity assays. Capped transcripts for seed inoculations were synthesized using the mMessage mMachine kit (Ambion), and uncapped transcripts were synthesized with the same kit by substituting a solution containing 10 mM each of ATP, GTP, CTP and UTP for the 2× NTP/CAP mixture. Seeds of the sweet corn variety Spirit were inoculated by vascular puncture (Redinbaugh et al., 2001). RNAs were quantitated using Ribogreen (Molecular Probes, Eugene, Oreg.) (Jones et al., 1998), and adjusted to the same concentrations. Transcript RNA (0.5 to 1.1 μg in water) was pipetted onto a presoaked seed and immediately inoculated to control for exposure to RNases on seed surfaces. Non-inoculated plants served as negative controls, and positive controls were inoculated with extract from MNeSV-infected maize leaves ground in 10 mM potassium phosphate, pH 7. Plants were scored for symptoms twice between 6 and 20 days after inoculation.

In vitro translations with wheat germ extract (WGE). Capped and uncapped RNAs were made from full length (SmaI-linearized) or truncated (Asp718I-linearized) templates of p2-2, pNsg1, pNsg2, and full length pNsg2-KLC. Translation reactions (25 μl) contained 0.5 pmol RNA, 10 μCi of 35S-methionine, 130 mM potassium acetate and 50% WGE (Promega). For some experiments 0.5 pmol of t2-2, tNsg1, and tNsg2 were mixed and used in the same 25 μl reaction. One-fifth of each reaction was heat denatured, separated on a 12% acrylamide Laemmli gel with a 6% stacking gel and electroblotted to nitrocellulose (Bolt and Mahoney, 1997). ¹⁴C-labeled proteins (Invitrogen) were used as molecular weight markers. Dried blots were exposed to a phosphorimager and analyzed with MultiAnalyst (BioRad) or OptiQuant (Perkin Elmer, Wellesley, Mass.) software.

Computer analysis. Sequence data was analyzed using Sequencher 3.1 (Gene Codes, Ann Arbor, Mich.) and the ClustalW program in MacVector 7.0 (Accelrys, San Diego, Calif.) was used for protein and RNA alignments. The 5′ and 3′ UTRs were analyzed with mfold (Zuker, 2003) online at http://www.bioinfo.rpi.edu/applications/mfold/old/rna/form1.cgi using both the version 3.1 free energy parameters (Mathews et al., 1999) which are fixed at 37° C., and version 2.3 free energy parameters (Walter et al., 1994) at 28° C. and 37° C. Folding predictions using the STAR program (v. 4) (Gultyaev et al., 1995) were performed using the greedy, stochastic, and genetic algorithms.

### Results

MNeSV genome sequence and secondary structure. Analysis of the initial sequence data for MNeSV suggested that about 10% of the viral sequence was missing based on the vRNA size estimate. Two approaches were used to complete the sequence of the 5′ end of the MNeSV genome. First, cDNAs synthesized with RNA ligase-mediated rapid amplification of cDNA ends (RLM-RACE) of vRNA that was not pretreated with calf intestinal phosphatase (CIP) or tobacco acid pyrophosphatase (TAP) were cloned into a plasmid vector and sequenced (Table 2). No cDNA could be amplified from vRNA that was pretreated with CIP followed by TAP before the RNA ligation reaction (data not shown) indicating that the vRNA was uncapped. Four cDNAs with the same sequence and one with a deletion of at least two A's corresponding to the 3′ end of the RNA oligomer, which terminated with GAAA, were identified (Table 2). This data suggested the RNA oligomer was partially deteriorated. Using an alternative strategy for RACE, cDNA synthesized from vRNA was C-tailed then used for PCR. The sequence of these cloned PCR products indicated the 5′ viral sequence was AGAUAU (Table 2). This sequence was consistent with results of primer extension analysis which produced cDNAs one base longer than the GATAT found in RLM-RACE derived clones (FIG. 1). Thus, it is likely that the RNA primer or the vRNA was degraded prior to ligation in the clones derived from RLM-RACE, and that the 5′ sequence of the MNeSV RNA is AGAUAU. This sequence is consistent with that of many tombusviruses (White and Nagy, 2004). The sequences of the 5′ RACE products indicated there were 28 additional nt at the 5′ end of the viral genome relative to the previously published sequence (Louie et al., 2000). In addition, sequences of the RACE products indicated that the residue at nt 88 of the full-length sequence was a C rather than the U reported earlier.

TABLE 2

Sequences of the 5′ end of the MNeSV vRNA
determined using RACE

| | # | | |
|---|---|---|---|
| Method ^{a} | clones ^{b} | Sequence ^{c} | SEQ ID NO: |
| RLM-RACE | 4 | atgaaaGATAT | (SEQ ID NO: 5) |
| RLM-RACE | 1 | atgaGATAT | |
| 5′ RACE | 2 | ggggggAGATAT | (SEQ ID NO: 6) |
| Tombusyirus | | AGAAAU | |
| consensus ^{d} | | | |

^{a} The sequences of cDNAs were obtained using the RLM-rACE kit (Ambion) with untreated vRNA or a 5′ RACE kit (Invitrogen) using total RNA.
^{b} The number of cDNAs showing the indicated sequence.
^{c} Sequences corresponding to viral RNA are indicated in uppercase letters, and those corresponding to the RNA oligomer or complement of the C-tail are indicated in lower case letters.
^{d} the dicot tombusvirus consensus vRNA 5′ terminal sequence (White and Nagy, 2004)

The initial MNeSV sequence data terminated within ORF 5, indicating that some of the coding region and the 3′UTR were missing. The 3′ end of the MNeSV genome was obtained using anchored cDNA cloning (Weng and Xiong, 1995) with an upstream primer corresponding to nt 3623-3647 of the previously published MNeSV sequence (nt 3651-3675 of the complete sequence, GenBank AF266518). Three cDNAs were sequenced, and two positions showed transitions at nt 3909 (A/G) or 3988 (C/T). Analysis of the resulting data indicated that the previously published partial sequence contained a duplicated fragment (nt 2188-2290 of the viral genome) inserted after nt 3832. After accounting for the duplication, 258 nt of additional sequence was identified in the anchored cDNA clones. Thus, the complete sequence of MNeSV is 4094 nt which is about 660 nt smaller than most dicot tombusviruses, and is 482 nt smaller than Cucumber Bulgarian latent virus (CBLV AY163842).

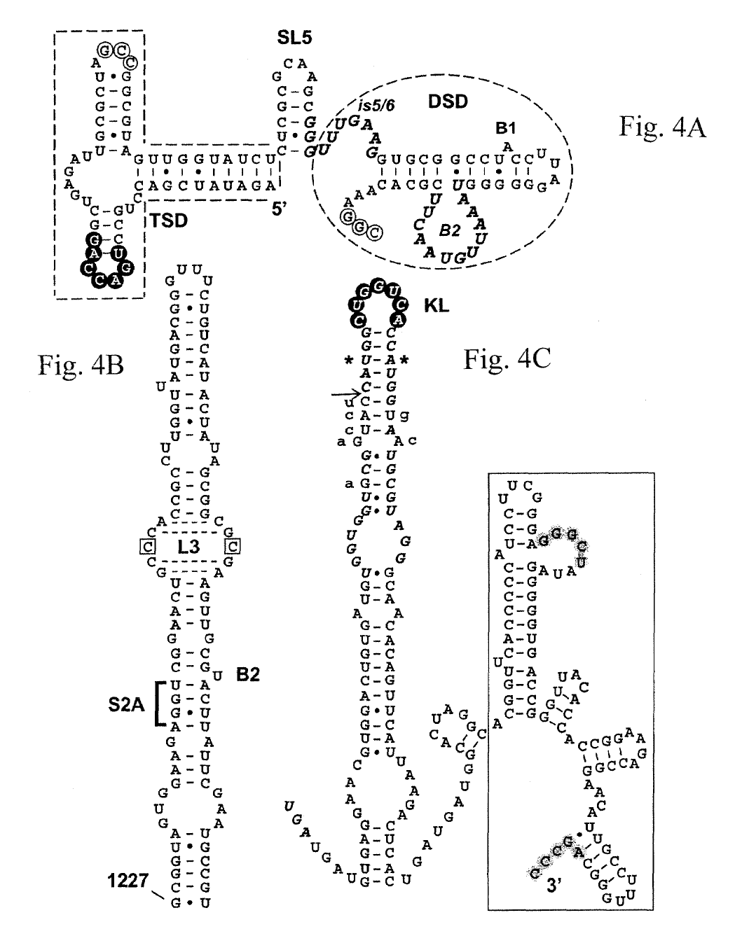

With the sequence completed, we reevaluated the positions and sizes of the ORFs, the similarity of encoded proteins and RNA, and predicted RNA secondary structures for comparison to other tombusviruses (FIG. 4 and FIG. 5A). The 122 nt MNeSV 5′UTR was similar in size to the 134 nt 5′UTR of CBLV, and was 44 nt shorter than that of TBSV-C M21958. ClustalW alignment of ten tombusviruses showed MNeSV's 5′UTR had 73% identity with CBLV, and lower similarity (45-57%) to other tombusviruses. The sequence similarity was greatest near the p30 start codon where identical stretches of 10 and 13 nt were found in MNeSV and all dicot tombusviruses (FIG. 4A and data not shown). RNA folding analysis was performed to determine whether MNeSV's 5′UTR contained structural features characteristic of a tombusvirus despite the low sequence similarity. RNA folding analysis using STAR (Gultyaev et al., 1995) and mfold (Zuker, 2003) predicted that the MNeSV 5′UTR contains secondary structures similar to those shown to be important for TBSV replication and predicted to be found in other tombusviruses (Ray et al., 2003; Wu et al., 2001) (FIG. 4 and FIG. 5A). The predicted structures include a 5′ T-shaped domain (TSD), a simple stem-loop (SL), a downstream domain (DSD), and a potential pseudoknot between the TSD loop and bases immediately preceding the p30 start codon (FIG. 4A). Although FIG. 4A represents the fourth lowest energy mfold structure (0.37 kcal/mol higher than lowest energy structure) it most closely resembles the TBSV structure, including identical single stranded sequences in the DSD that correspond to intervening sequence 5/6 (is5/6), bulge 1 (B1), and bulge 2 (B2) of TBSV (Ray et al., 2003). STAR predicted a similar structure (data not shown).

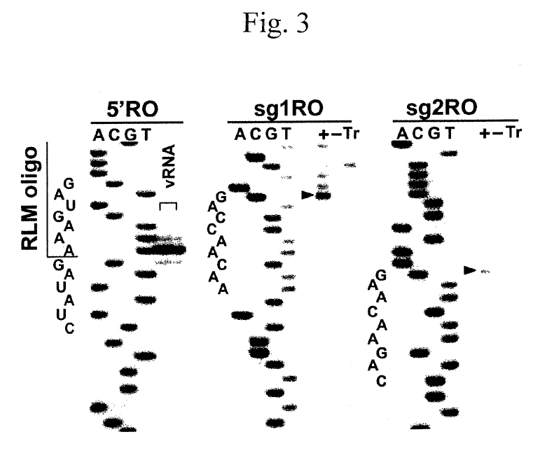

The RNA sequence of MNeSV ORF1 had low identity (43-59%) with ORF1 from dicot tombusviruses largely due to deletion(s) in MNeSV that occur about one third of the way (nt 407 to 434) from the 5′ end of the ORF (data not shown). ORFs 2, 4, and 5 of MNeSV showed 72-77% identity with their dicot tombusviral counterparts, bracketing the dissimilar necrovirus-like CP gene (ORF 3). The sizes of proteins expected from the first ORF and its readthrough product (ORF2) indicated that the 5′ portion of MNeSV encodes proteins of 30 and 88.6 kDa (FIG. 3A) instead of 33.3 and 92.1 kDa as originally reported (Louie et al., 2000). Additionally, the readthrough portion of p89 had 80-82% identity with the homologous protein regions encoded by dicot tombusviruses, giving an overall identity of 64-74% for p89 compared to other tombusviruses instead of the 40-42% originally reported. Within ORF2 the sequence (nt 1227-1324) corresponding to the internal replication element (IRE RII) of TBSV was predicted to fold into a long hairpin with loops and bulges (FIG. 2B). The structure included a CC mismatch which was critical for in vitro binding of TBSV p33 (Pogany et al., 2005) and replication of DI-RNAs in cucumber protoplasts (Monkewich et al., 2005) and yeast (Pogany et al., 2005). The MNeSV structure is more similar to the alternative structure predicted for TBSV since it included a bulge (B2) instead of a loop and the alternative base-pairing partners that form a four base-pair stem section (S2A) instead of a three base-pair section (Monkewich et al., 2005). The completed sequence of the 3′ end showed that the two nested ORFs encode proteins of 21.4 kDa and 19 kDa (FIG. 3A) with different carboxy-terminal sequences than originally reported. p21 had 68-73% sequence identity to the p22 proteins of tombusviruses while MNeSV's p19 had 54-59% identity with other tombusvirus p19 proteins as determined from ClustalW analysis (data not shown).

The 3′UTR was found to be 197 nt long, about 55% of the length of those found in most other tombusviruses. No ORF longer than 23 amino acids (nt 3891 to 3959) was found in this region. Compared to the 5′UTR, the 3′UTR of MNeSV showed less overall similarity to 3′UTRs of other tombusviruses largely due to the size differences, with the most similarity (51% identity) to CBLV using ClustalW analysis (data not shown). The 5′ half of the MNeSV 3′UTR was least similar to the same region in other tombusviral 3′UTRs, and there was no overall conservation of secondary structure, as this portion of the MNeSV 3′UTR was predicted to fold into a long stem-loop with four bulges (FIG. 4C). This differs from the Y-shaped structure predicted for most dicot tombusviruses and identified as the location of the 3′CITE of TBSV (Fabian and White, 2004). Using default program settings in mfold and STAR, none of the predicted structures for MNeSV contained a Y-shaped stem-loop (data not shown). A 44 nt sequence on the outer half of the MNeSV long SL showed 79% identity (FIG. 4C) to CBLV and 47-54% identity with the upstream arm region (SL-B) of the Y-shaped domain of the other dicot tombusviruses (data not shown). The seven nt of the terminal “kissing loop” (KL), which are complementary to nt in the first loop in the 5′UTR TSD (FIGS. 2A and C), are identical to CBLV and one nt different from the six nt loop of the other tombusviruses. The last 90 nt of MNeSV showed 65-77% sequence identity (ClustalW) to the terminal sequences of dicot tombusviruses making this the largest conserved region in either UTR. Both the STAR and mfold programs predicted stem-loop structures at the 3′ terminus similar to those mapped for TBSV or predicted for other tombusviruses (FIG. 4C). Furthermore, sequences and structures associated with the tombusvirus replication silencer element (RSE) are conserved (Fabian et al., 2003; Pogany, et al., 2003). Thus, there was remarkable conservation of motif order (FIG. 5A) and sequence between the MNeSV genome and those of other tombusviruses.

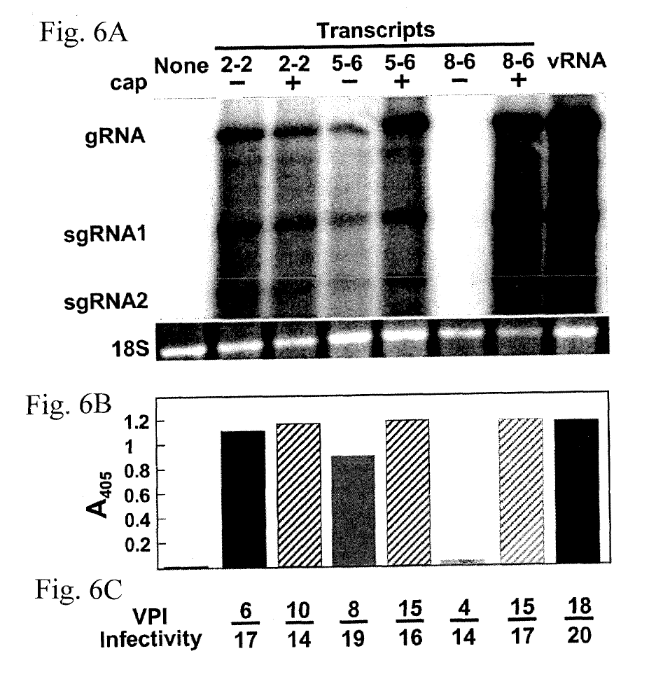

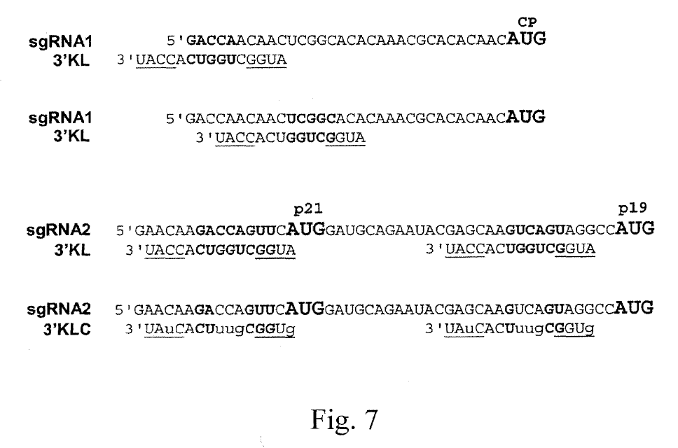

Identification of sgRNA transcription initiation sites and putative regulatory sequences. During tombusvirus replication, the CP is expressed from a large sgRNA and the two overlapping 3′ ORFs are expressed from a smaller sgRNA. Primer extension analysis of total RNA from MNeSV-infected and healthy maize plants indicated the presence of sgRNAs of 1607 and 781 nt initiating at nt 2488 and 3314, respectively (FIG. 3). Northern blot analysis of RNA from infected maize Black Mexican Sweet (BMS) suspension culture protoplasts confirmed the presence of two sgRNAs (FIG. 6). Complementary seven nt sequences that differ by one nt from activator sequence-1 (AS-1) and receptor sequence-1 (RS-1), two widely separated components that base pair as part of the sgRNA1 promoter for TBSV (Choi and White, 2002), were identified in MNeSV at nt 1476-1782 and nt 2478-2484 (FIG. 5A). Sequence comparisons indicated that the 11 nt immediately upstream of the sgRNA2 start site is identical to the TBSV sequences for RS-2 (nt 3303-3308) and core element-C (CE-C) (nt 3309-3313), which are essential for sgRNA2 accumulation in TBSV (Choi et al., 2001; Lin and White, 2004; Zhang et al., 1999). In addition, a sequence complementary to RS-2 and identical to the AS-2 of TBSV was found at nt 1436-1442. In TBSV these sequences comprise part of the sgRNA2 promoter (Lin and White, 2004). Complementary sequences in positions analogous to the TBSV sgRNA2 regulatory sequences distal element-A (DE-A) and CE-A (Choi et al., 2001; Zhang et al., 1999) were identified at nt 2510-2516 and 3293-3299, flanking the CP start and stop codons, respectively. Examination of the sgRNA 5′UTRs indicated they contain sequences complementary to the KL region in the 3′UTR (FIG. 7). For sgRNA1, two complementary sequences were found, and in sgRNA2, complementary sequences were identified just upstream of both the p21 and p19 start codons (FIG. 7). These results suggest that genomic elements required for transcription of MNeSV sgRNAs are similar to those of other tombusviruses.

Construction and infectivity of MNeSV transcription clones. Because of initial ambiguities regarding the 5′ nucleotide of MNeSV (Table 2) and sequence differences in the 3′UTR, plasmids representing six possible sequence variants were constructed such that each 5′ end was immediately downstream of a T7 RNA polymerase promoter and the 3′ end was upstream of a unique SmaI site (FIG. 5B and FIG. 5C). Three different 5′ end sequences were made based on the results of RACE experiments (FIG. 5C). The 3′ ends contained either a G and a C (p1-2, p4-3, p7-1) or an A and a T (p2-2, p5-6, p8-6 ) at nt 3909 and 3988, respectively, representing the variable bases found in the 3′ cDNAs. Transcripts synthesized in vitro from these cDNAs are identified throughout the text by replacing the “p” in the plasmid name with a “t”.

To determine the infectivity of the cDNAs, uncapped transcripts were synthesized and used to inoculate BMS protoplasts. The protoplasts were analyzed for accumulation of viral RNAs and CP 48 hours post-inoculation (hpi). Transcripts initiating with GAUAU and AGAUAU (t1-2, t2-2, t4-3, and t5-6) were readily infectious in protoplasts as shown by the accumulation of gRNA, sgRNAs and CP, and there was no apparent effect of the base changes in the 3′UTRs on the accumulation of MNeSV RNAs in protoplasts (FIG. 6 and data not shown). Inoculation of protoplasts with transcripts initiating with GGAUAU (t7-1 and t8-6) produced extremely low but detectable CP accumulation (FIG. 6 and data not shown). Additionally, sgRNAs as well as gRNA were visible for t7-1 and t8-6 inoculations when the phosphorimage data was compressed to levels that “overexpose” the images for all other positive RNA signals (data not shown), indicating very low levels of replication occurred.

Initial VPI inoculations of maize seeds were performed with RNAs generated from transcription reactions containing m7GpppGTP, and all six transcripts produced similar numbers of infected plants (FIG. 6 and data not shown). Our results with RLM-RACE showed that the MNeSV vRNA, like dicot tombusviruses, is not capped. To further examine the effect of capping on the infectivity of MNeSV, capped and uncapped transcripts were used to inoculate both protoplasts and seeds. Inoculation of protoplasts with capped and uncapped t2-2, t5-6, and t8-6 showed that although uncapped t8-6 was infectious at very low levels, inoculation with capped t8-6 increased the infectivity to levels seen with capped t2-2 and t5-6 (FIG. 6). Capped t2-2 and t5-6 were only moderately more infectious than their uncapped transcripts (FIG. 6 and data not shown). Note that t5-6 capped transcription reactions produced a mixture of uncapped t5-6 and capped t2-2 RNAs.

To determine whether capped and uncapped versions of t2-2, t5-6, and t8-6 showed differential infectivity in plants as well as protoplasts, capped and uncapped transcripts were inoculated to maize seeds via VPI. All plants that became infected showed the same rate of symptom appearance and symptom severity, regardless of whether they were inoculated with transcripts or MNeSV-infected plant extract. Both uncapped and capped transcripts of all three cDNAs infected plants but capped transcripts infected a larger fraction (FIG. 6). While differences in the infectivity in capped vs. uncapped transcripts were apparent after protoplast inoculation and VPI, the difference in infectivity of capped vs. uncapped t8-6 was less after VPI. This may reflect the effect of the time of analysis or the relatively small numbers of samples analyzed.

To ensure that the observed differences in infectivity of p8-6 was not due to unexpected mutations, inserts of both p2-2 and p8-6 were completely sequenced to confirm that the only difference between them was the extra G at the 5′ end of the p8-6 insert. Further, the differential replication of the capped and uncapped forms of t8-6 in protoplasts was confirmed using inocula derived from different plasmid and transcript preparations (data not shown). This data indicated that in protoplasts, uncapped transcripts initiating with GGAUAU were much less infectious than transcripts initiating with GAUAU and AGAUAU, and all capped transcripts had similar infectivity which was slightly better than uncapped t2-2 and t5-6 infectivity. p2-2 was selected as the “wild type” transcription cDNA for MNeSV since uncapped t2-2 was highly infectious in protoplasts and plants, and its 5′G allowed for more efficient in vitro synthesis compared to the equally infectious p5-6. Thus, uncapped transcripts from p2-2 contain nt 2-4094 of the Genbank (AF266518) sequence. These data indicate that, like tombusvirus transcripts, uncapped MNeSV transcripts are infectious in plants. Further, there is some lack of discrimination for the 5′ nucleotide of the infectious transcripts in plants, but in protoplasts, the infectivity of uncapped transcripts initiating with GG is greatly reduced.

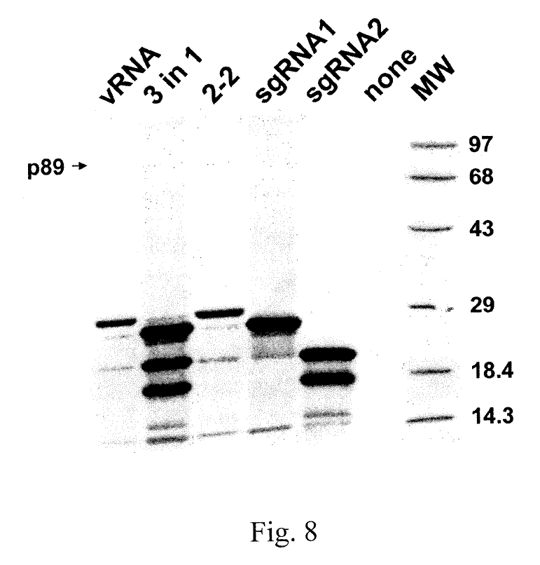

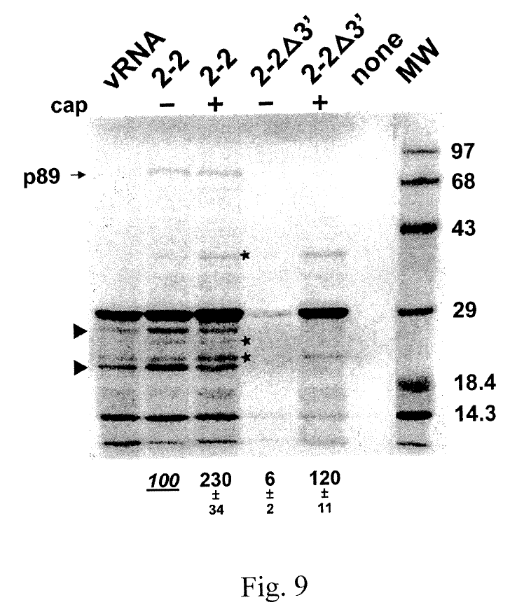

In vitro synthesis of viral proteins from MNeSV transcripts. To confirm the translation strategy and determine the relative mobility of the proteins encoded in MNeSV, transcripts for gRNA and sgRNAs were synthesized and translated in vitro. Plasmids pNsg1 and pNsg2 encoding sgRNA1 or sgRNA2, respectively, downstream of a T7 RNA polymerase promoter were constructed and used to synthesize sgRNA transcripts with the subgenomic 5′ and 3′ ends determined in earlier experiments. Equimolar amounts of vRNA, uncapped t2-2, and uncapped sgRNA transcripts were used for in vitro translations in WGE in the presence of ³⁵S-methionine, and equal volumes of the reactions were analyzed by SDS-PAGE (FIG. 8 and FIG. 9). The five MNeSV-encoded proteins migrated with apparent molecular weights within 5-10% of the molecular weights calculated from the amino acid composition. The major product of vRNA and t2-2 was the 30 kDa protein, and in vitro translation of t2-2 also produced p89 at about 2% of the level of p30. p89 was not detected in any in vitro translation using vRNA (FIG. 9 and data not shown). Lack of p89 synthesis from vRNA may have been due to the presence of an inhibitor that copurified with vRNA or a smaller fraction of full length RNA in vRNA preparations compared to transcripts. CP and p21 were minor products in translation reactions programmed with vRNA and t2-2, as were proteins corresponding in size to those expected from initiation at some internal methionines in ORFs 1 and 2 (FIG. 8 and FIG. 9 and data not shown). The primary translation product from tNsg1 was the 27 kDa CP, while tNsg2 produced 21 kDa and 19 kDa proteins as expected (FIG. 8). These data are consistent with MNeSV encoding five proteins of the sizes predicted from the ORFs.

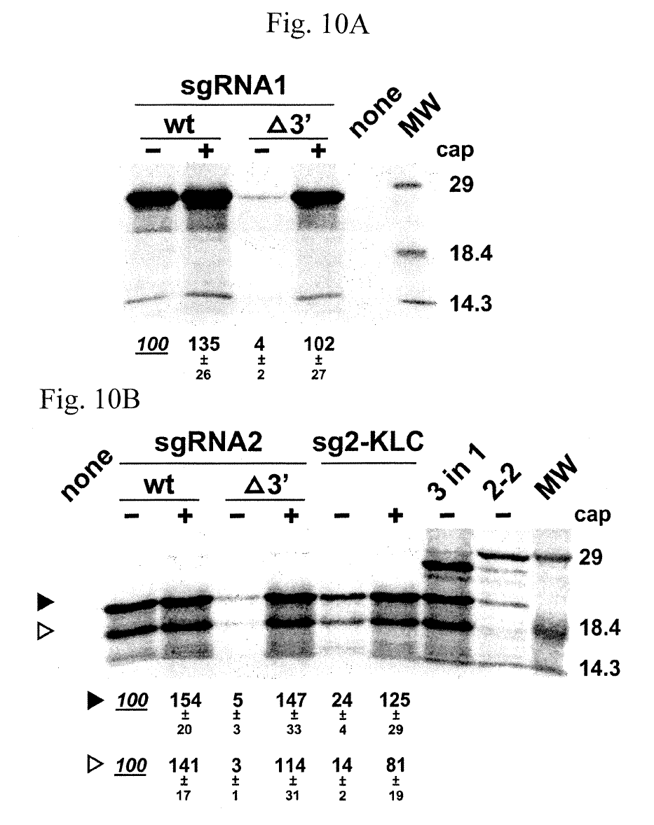

Cap-independent in vitro translation of MNeSV transcripts in wheat germ extracts. WGE shows a marked discrimination for translation of most capped RNAs compared to their uncapped counterparts, but some CITEs functionally replace the ^{m}7GpppN cap. To determine whether MNeSV encodes a 3′CITE that is active in WGE, in vitro translations of the capped and uncapped t2-2 RNAs were compared with translations of transcripts truncated to remove the KL by digestion of p2-2 with Asp718I prior to transcription (t2-2Δ3′) (FIG. 4C and FIG. 5B). More than twice as much p30 was synthesized when capped t2-2 was translated compared to translation of uncapped t2-2 (FIG. 9). Truncated uncapped transcripts produced 8-50 fold less p30 than uncapped t2-2 (FIG. 9). In contrast, translation of capped t2-2Δ3′ produced about 50% as much p30 as capped t2-2. These data showed that the 3′ 156 nt contains a CITE that functionally replaces a 5′ cap, and suggested that it may also provide an additional function, possibly as a poly(A) replacement (Guo et al., 2000). The activity of the 3′CITE was also tested for sgRNA translations. Similarly to the full length transcripts, translation rates of capped sgRNA transcripts were higher than translation of uncapped sgRNA transcripts (FIG. 10). However, the extent of the increase was smaller than for t2-2. Removing the 3′ ends decreased translation of uncapped sgRNA transcripts similarly to the decrease in t2-2Δ3′ translations, and capping the truncated sgRNA transcripts partially relieved the decrease indicating that the 3′CITE also functions on the sgRNAs (FIG. 10). To determine if a mutation in the KL could inactivate the 3′CITE in sgRNA2, pNsg2-KLC was constructed. Transcripts from this plasmid contained changes in five nt in or near the KL that would decrease the number of potential consecutive base pairs from eight to three and from six to two for the predicted pairing sequences preceding the p21 and p19 start codons, respectively (FIG. 7). Mfold analysis of the 3′UTR of pNsg2-KLC predicted the same structure as for MNeSV (data not shown). The levels of p21 and p19 translation products from uncapped tNsg2-KLC were about four- and seven-fold lower, respectively, compared to products from tNsg2, and capping increased the translation rates (FIG. 10). Thus, the MNeSV genome has a 3′CITE that functions on gRNA and sgRNAs, and the KL is likely to be an important part of the 3′ CITE.

### Discussion

The completion of the sequence of MNeSV indicated that it is most closely related to tombusviruses. The nonstructural proteins are clearly most closely related to their dicot tombusviral homologs. Sequence comparisons, phylogenetic analysis, and RNA folding analysis of MNeSV RNA predict the formation of secondary structures and long-distance base-pairing interactions similar or identical to those previously shown to be involved with regulation of TBSV replication, sgRNA synthesis, and translation. These structures include TSD, SL5, and DSD located in the 5′UTR (Ray et al., 2003; Wu et al., 2001), IRE RII (Monkewich et al., 2005), sgRNA1 regulatory elements (AS1, SL1sg1, RS1) (Choi and White, 2002), sgRNA2 regulatory elements (AS2, RS2, DE, CE) (Lin and White, 2004), and the 3′ terminal hairpins containing the RSE (Fabian et al., 2003; Pogany et al., 2003) (FIG. 5A). The predicted secondary structure of the MNeSV 3′CITE differs from that for most tombusviruses, but it is located in the equivalent region of the 3′UTR and contains a loop sequence complementary to sequences in the TSD and 5′UTRs of the sgRNAs similar to TBSV.

The ability of the 3′CITE to function in vitro, the lack of infectivity by leaf rub inoculation, and the homology of the CP to those of necroviruses are characteristics of MNeSV that distinguish it from the dicot-infecting tombusviruses. Recent work indicates that, while vesicles form in MNeSV-infected maize cells, they are not the same as the multivesicular bodies typically found in dicot cells after tombusvirus infection (De Stradis et al., 2005). Of these differences, the activity of the 3′CITE in WGE, the lack of transmission by rub inoculation, and cellular structures associated with virus infection in vivo may reflect the differences between monocot and dicot hosts rather than differences between MNeSV and tombusviruses. The closer relationship of the MNeSV CP to necroviruses than tombusviruses may be the result of recombination between a tombusvirus and a necrovirus, since otherwise the CP ORF would have had to undergo a deletion of the region encoding the protruding domain as well as extensive mutation within the remainder of the ORF to produce the MNeSV CP. It is interesting to note that ClustalW alignments of MNeSV RNA and dicot tombusvirus RNA sequences show that the borders of the MNeSV sequence with high tombusvirus homology surrounding the CP ORF precisely retain two tombusviral regulatory sequences; the first six nt of sgRNA1, which is a proposed base-pairing partner for the 3′CITE KL (FIG. 7), and RS-2 located 11 nt upstream of the sgRNA2 start site, the proposed base-pairing partner for AS-2 (FIG. 5A and data not shown). Retention of these sequences suggests that MNeSV uses the same or similar mechanisms as those identified in TBSV for regulating translation and sgRNA synthesis. Further support for placing MNeSV in the genus Tombusvirus comes from a phylogenetic analysis of the family Tombusviridae based on whole genome protein sequences that definitively placed MNeSV as a tombusvirus (Stuart et al., 2004). Taken together, the results indicate that, if MNeSV is not classified as a tombusvirus, it should be identified as the type member of a new genus within the family Tombusviridae.

The relative infectivity of the MNeSV transcripts with three different 5′ ends differed when tested in plants and protoplasts, and comparison with results from other tombusviral systems may help elucidate these results. Infectious transcripts have been made from cDNAs for six dicot-infecting tombusviruses, and all were initially tested for infectivity in plants. The Cucumber necrosis virus (CNV M25270) cDNA produces a transcript initiating with GAAAU (Rochon and Johnston, 1991) which is similar to the MNeSV cDNA p2-2. cDNAs for TBSV-C (Hearne et al., 1990), Cymbidium ringspot virus (CyRSV X15511) (Dalmay et al., 1993), Artichoke mottle crinkle virus (AMCV X62493) (Tavazza et al., 1994), Carnation Italian ringspot virus X85215 (Burgyan et al., 1996), and TBSV-P U80935 (Szittya et al., 2000) encode transcripts initiating with GGAAAU instead of the AGAAAU identified as the 5′ vRNA sequence (Burgyan et al., 1996; Szittya et al., 2000), deduced as the 5′ sequence from DI-RNAs (Dalmay et al., 1995; Finnen and Rochon, 1995) or by comparison to known tombusviral 5′ ends. Thus, the cDNAs for these viruses have a 5′ substitution similar to p8-6. As was found for seed inoculation of infectious MNeSV transcripts, relative infectivity of capped and uncapped transcripts of TBSV-C, CNV, CyRSV, and AMCV, the only transcripts tested this way, showed little or no difference (Dalmay et al., 1993; Hearne et al., 1990; Rochon and Johnston, 1991; Tavazza et al., 1994).

It was surprising that uncapped transcripts from p8-6 showed such poor replication in protoplasts, since its 5′ end was similar to transcripts for five of the dicot-infecting tombusvirus cDNAs. There are several possible explanations for the effect of capping on t8-6 infectivity in protoplasts. A cap structure may protect the transcripts from 5′ exonuclease activity during the inoculation procedure, thereby increasing the number of intact RNA molecules available for translation and replication. Since there was a much smaller increase in protoplast infectivity produced by capping t2-2 and t5-6, it seems unlikely that this protection can account for the large capping effect on t8-6 infectivity in protoplasts, but might explain the effect of capping on all transcripts in VPI infectivity (FIG. 6).

The lower infectivity of uncapped t8-6 may indicate it is a poor template for replication or it produces lower amounts of the RdRp from initial translation. The equally efficient synthesis of p30 and p89 from uncapped t8-6 and t2-2 in WGE (Scheets, data not shown), suggests that poor translation of the RdRp ORFs is not likely to explain the low infectivity. Conversely, capping t8-6 might increase its infectivity indirectly via translation. Since capped transcripts produced 1.5-3 times as much p30 and p89 as uncapped transcripts in WGE for both t2-2 (FIG. 8) and t8-6 (data not shown), a similar increase in initial RdRp synthesis in maize cells might increase initial replication enough to more rapidly overcome the disadvantage of having a less optimal 5′ end sequence, accumulating to high levels by 48 hpi. The 5′ ends of progeny derived from p8-6 transcripts are probably repaired during replication to the viral sequence as was seen for transcripts of a TBSV DI-RNA (Panavas and Nagy, 2003). The 5′ sequence of progeny vRNA from plants or protoplasts inoculated with transcripts was not determined. But if no repair occurred, all progeny from capped and uncapped t8-6 infections would look like uncapped t8-6, and replication would be expected to continue at the extremely poor rate seen at 48 hpi in protoplasts. The more similar infection rates of uncapped t2-2, t5-6, and t8-6 seen in plants suggests that the poor replication of uncapped t8-6 detected at 48 hpi in protoplasts is overcome soon after that since no difference in time of symptom appearance between the different transcripts was observed. Since the relative infectivity of capped and uncapped transcripts for the dicot-infecting tombusvirus cDNAs was not determined in protoplasts, it is not known whether the results seen with p8-6 transcripts are specific for MNeSV or is a general characteristic for tombusviruses.

Many positive strand RNA viruses that lack 5′ genome-linked proteins or caps have secondary structures that serve as translational enhancers such as the 3′CITE of TBSV (Fabian and White, 2004). Base pairing between residues in the TBSV 3′CITE and the TSD in the 5′UTR is required for the function of the TBSV 3′CITE, which is not active in WGE (Fabian and White, 2004; Wu and White, 1999). RNA secondary structure programs predicted no RNA secondary structure for MNeSV similar to the Y-shaped domain in the TBSV 3′UTR. This is not surprising considering the smaller size of the MNeSV 3′UTR and its lack of sequence similarity with the upstream portion of the TBSV 3′UTR. Nevertheless, the MNeSV 3′UTR contains a CITE which is active in WGE on gRNA and sgRNAs (FIG. 9 and FIG. 10). Both the 5′ and 3′ UTRs of MNeSV were most similar to those of CBLV, and the putative CBLV 3′CITE region (Fabian and White, 2004) is predicted by STAR to fold into a six bp-seven nt loop (data not shown) very similar to the terminus of the MNeSV 3′CITE (FIG. 4C). Interestingly, the seven nt loop in MNeSV has potential base-pairing partners in the 5′UTRs of both the gRNA and the sgRNAs (FIG. 7), and removal of this region abolished in vitro translational enhancing activity. It is possible that removing the 3′ terminal 156 nt greatly decreased the stability of transcripts leading to decreased protein synthesis. However, if decreased transcript stability was the sole cause of decreased protein synthesis, the translation of both capped and uncapped truncated transcripts would be expected to decrease by similar amounts compared to their full length transcripts. Instead, truncated capped transcripts were translated an order of magnitude more efficiently than truncated uncapped transcripts (FIG. 9 and FIG. 10). Thus, while the 3′UTR of MNeSV differs substantially in structure from that of TBSV, it is likely to carry a functionally similar translational enhancer.

The MNeSV 3′CITE shares some characteristics with other, well-studied, viral translational enhancers. BYDV-PAV, a luteovirus closely related to some members of the family Tombusviridae (Miller et al., 2002), has a 3′TE (BTE) consisting of a 109 nt cruciform structure (Guo et al., 2000) that base-pairs with sequences in the 5′UTRs of gRNA and sgRNA1 to function in vitro (Guo et al., 2001). The MNeSV 3′CITE does not contain the highly conserved 18 nt sequence nor the secondary structure of BTE, and in contrast to BYDV (Allen et al., 1999), capped MNeSV gRNA transcripts do not show decreased infectivity in protoplasts. However, the smaller relative decrease in protein synthesis for the mutant transcript tNsg2-KLC compared to tNsg2Δ3′ and the stronger translation of capped full-length transcripts compared to capped Δ3′ transcripts are similar to BYDV. This suggests that the 3′UTR contains additional translation functions such as a poly(A) replacement similar to BYDV-PAV (Guo et al., 2000). After accounting for methionine content, the translation of equimolar amounts of gRNA and sgRNA transcripts in individual reactions indicates a translation efficiency of sgRNA2>sgRNA1>gRNA (FIG. 8). This may indicate that the 5′UTRs of the sgRNAs interact with the 3′CITE and/or translation factors more strongly than does the gRNA 5′UTR as is the case for BYDV-PAV (Wang et al., 1999). Alternatively, the 5′-3′ interaction required for 3′CITE activity may form more readily on shorter RNAs. A stronger 3′CITE activity for sgRNAs than gRNA was also suggested by the smaller increases in translation produced by capping full length sgRNAs than for capping t2-2 (FIG. 9 and FIG. 10). When equimolar amounts of gRNA and sgRNAs were translated together, the level of p30 synthesis was reduced to about one fourth the amount produced from translation of an equal amount of t2-2 alone. In contrast, the levels of sgRNA-encoded proteins were the same in mixed and single translations (3 in 1, FIG. 10 and data not shown). The decrease in gRNA translation in the mixed translations was not due to limiting amounts of initiation factors since sgRNA translations did not decrease. These results suggest that one or both MNeSV sgRNAs might inhibit initiation on gRNA as was seen with sgRNA2 inhibition on gRNA and sgRNA1 of BYDV-PAV (Wang et al., 1999).

The predicted structure of the MNeSV 3′CITE shares a general similarity with the predicted structure of the translational enhancer domain (TED) found in the 3′UTR of STNV in that they both contain a long stem-loop with various bulges (van Lipzig et al., 2002). There is no sequence similarity or similarity in the sizes and locations of bulges and stem lengths, and currently there is no indication that the general structural similarity will be relevant at the functional level.

The construction of an infectious transcript cDNA of MNeSV provides a tool for future analysis of many different aspects of the MNeSV life cycle in a monocot host. Analysis of viral protein function is underway. Studies to determine the molecular basis of the difference in infectivity of t8-6 and t2-2 may shed light on viral replication processes in maize. In addition, chemical analysis of the MNeSV 3′CITE secondary structure, determination of long distance base-pairing with 5′UTRs in vivo and in vitro, and identification of interacting factors from WGE will provide insight into the mechanisms and regulation of the translational process. These experiments will allow comparisons with both the TBSV 3′CITE and the well characterized 3′TEs of BYDV and STNV.

### REFERENCES

Allen, E. M., Wang, S., Miller, W. A., 1999. Barley yellow dwarf virus RNA requires a cap-independent translation sequence because it lacks a 5′ cap. Virology 253, 139-144. doi:10.1006/viro. 1998.9507
Burgyan, J., Rubino, L., Russo, M., 1991. De novo generation of Cymbidium ringspot virus defective interfering RNA. J. Gen. Virol. 72, 505-509.
Burgyan, J., Rubino, L., Russo, M., 1996. The 5′-terminal region of a tombusvirus genome determines the origin of multivesicular bodies. J. Gen. Virol. 77, 1967-1974.
Choi, I.-R., Ostrovsky, M., Zhang, G., White, K. A., 2001. Regulatory activity of distal and core RNA elements in tombusvirus subgenomic mRNA2 transcription. J. Biol. Chem. 276, 41761-41768. doi: 10.1074/jbc.M106727200
Choi, I.-R., White, K. A., 2002. An RNA activator of subgenomic mRNA1 transcription in Tomato bushy stunt virus. J. Biol. Chem. 277, 3760-3766. doi:10.1074/jbc.M109067200
Chu, M., Desvoyes, B., Turina, M., Noad, R., Scholthof, H. B., 2000. Genetic dissection of Tomato bushy stunt virus p19-protein-mediated host-dependent symptom induction and systemic invasion. Virology 266, 79-87. doi:10.1006/viro.1999.0071
Dalmay, T., Russo, M., Burgyan, J., 1993. Repair in vivo of altered 3′ terminus of Cymbidium ringspot tombusvirus RNA. Virology 192, 551-555. doi:10.1006/viro.1993.1071
Dalmay, T., Szittya, G., Burgyan, J., 1995. Generation of defective interfering RNA dimers of Cymbidium ringspot tombusvirus. Virology 207, 510-517. doi: 10.1006/viro.1995.1111
Danthinne, X., Seurinck, J., Meulewaeter, F., Van Montagu, M., Cornelissen, M., 1993. The 3′ untranslated region of Satellite tobacco necrosis virus-RNA stimulates translation in vitro. Mol. Cell. Biol. 13, 3340-3349.
De Stradis, A., Redinbaugh, M. G., Abt, J. J., Martelli, C. P. (2005) Ultrastructure of Maize necrotic streak virus infections. J. Plant Path. 87, 213-221.
Fabian, M. R., Na, H., Ray, D., White, K. A., 2003. 3′-terminal RNA secondary structures are important for accumulation of Tomato bushy stunt virus DI RNAs. Virology 313, 567-580. doi:10.1016/S0042-6822(03)00349-0
Fabian, M. R., White, K. A., 2004. 5′-3′ RNA-RNA interaction facilitates cap- and poly(a) tail-independent translation of Tomato bushy stunt virus mRNA: A potential common mechanism for Tombusviridae. J. Biol. Chem. 279, 28862-28872. doi:10.1074/jbc.M401272200
Finnen, R. L., Rochon, D. M., 1995. Characterization and biological activity of DI RNA dimers formed during Cucumber necrosis virus coinfections. Virology 207, 282-286. doi:10.1006/viro.1995.1078
Gultyaev, A. P., van Batenburg, F. H., Pleij, C. W., 1995. The computer simulation of RNA folding pathways using a genetic algorithm. J. Mol. Biol. 250, 37-51. doi:10.1006/jmbi.1995.0356
Guo, L., Allen, E. M., Miller, W. A., 2000. Structure and function of a cap-independent translation element that functions in either the 3′ or the 5′ untranslated region. RNA 6, 1808-1820.
Guo, L., Allen, E. M., Miller, W. A., 2001. Base-pairing between untranslated regions facilitates translation of uncapped, nonpolyadenylated viral RNA. Molecular Cell 7, 1103-1109. doi:10.1016/S1097-2765(01)00252-0
Havelda, Z., Hornyik, C., Crescenzi, A., Burgyan, J., 2003. In situ characterization of Cymbidium ringspot tombusvirus infection-induced posttranscriptional gene silencing in Nicotiana benthamiana. J. Virol. 77, 6082-6086. doi:10.1128/JVI.77.10.6082-6086.2003
Hearne, P. Q., Knorr, D. A., Hillman, B. I., Morris, T. J., 1990. The complete genome structure and synthesis of infectious RNA from clones of Tomato bushy stunt virus. Virology 177, 141-151. doi:10.1016/0042-6822(90)90468-7
Hurst, R., Schenborn, E., Shultz, J., Kar, S., Leippe, D., and Creswell, D. 2005. Higher yields plus single-tube format. Promega Notes #90, May 2005 19-21.
Jones, L. J., Yue, S. T., Cheung, C. Y., Singer, V. L., 1998. RNA quantitation by fluorescence-based solution assay: Ribogreen reagent characterization. Anal. Biochem. 265, 368-374. doi:10.1006/abio.1998.2914
Kawaguchi, R., Bailey-Serres, J., 2002. Regulation of translational initiation in plants. Curr. Opin. Plant Biol. 5, 460-465. doi:10.1016/S1369-5266(02)00290-X
Koh, D. C.-Y., Liu, D. X., Wong, S.-M., 2002. A six-nucleotide segment within the 3′ untranslated region of Hibiscus chlorotic ringspot virus plays an essential role in translational enhancement. J. Virol. 76, 1144-1153. doi: 10.1128/JVI.76.3.1144-1153.2002
Lin, H.-X., White, K. A., 2004. A complex network of RNA-RNA interactions controls subgenomic mRNA transcription in a tombusvirus. EMBO J. 23, 3365-3374. doi:10.1038/sj.emboj.7600336
Lommel, S. A., Martelli, G. P., Rubino, L., Russo, M., 2005a. Genus Necrovirus. In: (Fauquet, C. M., Mayo, M. A., Maniloff, J., Desselberger, U., Ball, L. A., Eds.), Virus taxonomy: Classification and nomenclature of viruses. Eighth report of the international committee on taxonomy of viruses. Elsevier Academic Press, San Diego, pp. 926-929.
Lommel, S. A., Martelli, G. P., Rubino, L., Russo, M., 2005b. Genus Tombusvirus. In: (Fauquet, C. M., Mayo, M. A., Maniloff, J., Desselberger, U., Ball, L. A., Eds.), Virus taxonomy: Classification and nomenclature of viruses. Eighth report of the international committee on taxonomy of viruses. Elsevier Academic Press, San Diego, pp. 914-917.
Louie, R., Redinbaugh, M., Gordon, D., Abt, J., Anderson, R., 2000. Maize necrotic streak virus, a new maize virus with similarity to species of the family Tombusviridae. Plant Dis. 84, 1133-1139.
Mathews, D. H., Sabina, J., Zuker, M., Turner, D. H., 1999. Expanded sequence dependence of thermodynamic parameters improves prediction of RNA secondary structure. J. Mol. Biol. 288, 911-940. doi:10.1006/jmbi.1999.2700
McLean, M. A., Campbell, R. N., Hamilton, R. I., Rochon, D. M., 1994. Involvement of the Cucumber necrosis virus coat protein in the specificity of fungus transmission by Olpidium bornovanus. Virology 204 (2). 1994 p. p.840-842, 840. doi:10.1006/viro.1994.1604
Meulewaeter, F., Danthinne, X., Van Montagu, M., Cornelissen, M., 1998. 5′- and 3′-sequences of Satellite tobacco necrosis virus RNA promoting translation in tobacco. Plant J. 14, 169-176. doi:10.1046/j.1365-313X.1998.00104.x
Meulewaeter, F., van Lipzig, R., Gultyaev, A. P., Pleij, C. W. A., Van Damme, D., Cornelissen, M., van Eldik, G., 2004. Conservation of RNA structures enables TNV and BYDV 5′ and 3′ elements to cooperate synergistically in cap-independent translation. Nucleic Acids Res. 32, 1721-1730. doi:10.1093/nar/gkh338
Miller, W. A., Liu, S., Beckett, R., 2002. Barley yellow dwarf virus: Luteoviridae or Tombusviridae? Molec. Plant Path. 3, 177-183. doi:10.1046/j.1364-3703.2002.00112.x
Mizumoto, H., Tatsuta, M., Kaido, M., Mise, K., Okuno, T., 2003. Cap-independent translational enhancement by the 3′ untranslated region of Red clover necrotic mosaic virus RNA1. J. Virol. 77, 12113-12121. doi: 10.1128/JVI.77.22.12113-12121.2003
Monkewich, S., Lin, H.-X., Fabian, M. R., Xu, W., Na, H., Ray, D., Chernysheva, O. A., Nagy, P. D., White, K. A., 2005. The p92 polymerase coding region contains an internal RNA element required at an early step in tombusvirus genome replication. J. Virol. 79, 4848-4858. doi:10.1128/JVI.79.8.4848-4858.2005
Nagy, P. D., Pogany, J., 2000. Partial purification and characterization of Cucumber necrosis virus and Tomato bushy stunt virus RNA-dependent RNA polymerases: Similarities and differences in template usage between tombusvirus and carmovirus RNA-dependent RNA polymerases. Virology 276, 279-288. doi:10.1006/viro.2000.0577
Panavas, T., Nagy, P. D., 2003. Yeast as a model host to study replication and recombination of defective interfering RNA of Tomato bushy stunt virus. Virology 314, 315-325. doi:10.1016/S0042-6822(03)00436-7
Panavas, T., Pogany, J., Nagy, P. D., 2002. Analysis of minimal promoter sequences for plus-strand synthesis by the Cucumber necrosis virus RNA-dependent RNA polymerase. Virology 296, 263-274. doi:10.1006/viro.2002.1423
Pantaleo, V., Rubino, L., Russo, M., 2003. Replication of Carnation Italian ringspot virus defective interfering RNA in Saccharomyces cerevisiae. J. Virol. 77, 2116-2123. doi: 10.1128/JVI.77.3.2116-2123.2003
Pogany, J., Fabian, M. R., White, K. A., Nagy, P. D., 2003. A replication silencer element in a plus-strand RNA virus. EMBO J. 22, 5602-5611. doi:10.1093/emboj/cdg523
Pogany, J., White, K. A., Nagy, P. D., 2005. Specific binding of tombusvirus replication protein p33 to an internal replication element in the viral RNA is essential for replication. J. Virol. 79, 4859-4869. doi:10.1128/JVI.79.8.4859-4869.2005
Qiu, W., Park, J. W., Scholthof, H. B., 2002. Tombusvirus p19-mediated suppression of virus-induced gene silencing is controlled by genetic and dosage features that influence pathogenicity. MPMI 15, 269-280.
Qu, F., Morris, T. J., 2000. Cap-independent translational enhancement of Turnip crinkle virus genomic and subgenomic RNAs. J. Virol. 74, 1085-1093.
Ray, D., Wu, B., White, K. A., 2003. A second functional RNA domain in the 5′UTR of the Tomato bushy stunt virus genome: Intra- and interdomain interactions mediate viral RNA replication. RNA 9, 1232-1245. doi/10.1261/rna.5630203
Reade, R., Wu, Z., Rochon, D., 1999. Both RNA rearrangement and point mutation contribute to repair of defective chimeric viral genomes to form functional hybrid viruses in plants. Virology 258 (2). 1999 p. p.217-231, 217. doi:10.1006/viro.1999.9726
Redinbaugh, M. G., Louie, R., Ngwira, P., Edema, R., Gordon, D. T., Bisaro, D. M., 2001. Transmission of viral RNA and DNA to maize kernels by vascular puncture inoculation. J. Virol. Methods 98, 135-143. doi:10.1016/S0166-0934(01)00369-X
Rochon, D. M., Johnston, J. C., 1991. Infectious transcripts from cloned Cucumber necrosis virus cDNA: Evidence for a bifunctional subgenomic mRNA. Virology 181, 656-665. doi:10.1016/0042-6822(91)90899-M
Sambrook, J., Fritsch, E. F., Maniatis, T., Eds., 1989. Molecular cloning: A laboratory manual. Cold Spring Harbor: Cold Spring Harbor Laboratory Press.
Scheets, K., 2000. Maize chlorotic mottle machlomovirus expresses its coat protein from a 1.47-kb subgenomic RNA and makes a 0.34-kb subgenomic RNA. Virology 267, 90-101. doi:10.1006/viro.1999.0107
Scholthof, H. B., Scholthof, K. B., Kikkert, M., Jackson, A. O., 1995. Tomato bushy stunt virus spread is regulated by two nested genes that function in cell-to-cell movement and host-dependent systemic invasion. Virology 213, 425-438. doi:10.1006/viro.1995.0015
Scholthof, K. B., Scholthof, H. B., Jackson, A. O., 1995. The effect of defective interfering RNAs on the accumulation of Tomato bushy stunt virus proteins and implications for disease attenuation. Virology 211, 324-328. doi:10.1006/viro.1995.1410
Schubert, M., Brayton, P. R., Meier, E., 1988. Fusion of DNA sequences without the use of shared restriction sites. Focus 10, 8-9.
Shen, R., Miller, W. A., 2004. The 3′ untranslated region of Tobacco necrosis virus RNA contains a Barley yellow dwarf virus-like cap-independent translation element. J. Virol. 78, 4655-4664. doi:10.1128/JVI.78.9.4655-4664.2004
Stuart, G., Moffett, K., Bozarth, R. F., 2004. A whole genome perspective on the phylogeny of the plant virus family Tombusviridae. Arch. Virol. 149, 1595. doi:10.1007/s00705-004-0298-7
Szittya, G., Salamon, P., Burgyan, J., 2000. The complete nucleotide sequence and synthesis of infectious RNA of genomic and defective interfering RNAs of TBSV-P. Virus Res. 69, 131-136. doi:10.1016/S0168-1702(00)00178-7
Tavazza, M., Lucioli, A., Calogero, A., Pay, A., Tavazza, R., 1994. Nucleotide sequence, genomic organization and synthesis of infectious transcripts from a full-length clone of Artichoke mottle crinkle virus. J. Gen. Virol. 75, 1515-1524.
Timmer, R. T., Benkowski, L. A., Schodin, D., Lax, S. R., Metz, A. M., Ravel, J. M., Browning, K. S., 1993. The 5′ and 3′ untranslated regions of Satellite tobacco necrosis virus-RNA affect translational efficiency and dependence on a 5′ cap structure. J. Biol. Chem. 268, 9504-9510.
van Lipzig, R., Gultyaev, A. P., Pleij, C. W. A., van Montagu, M., Cornelissen, M., Meulewaeter, F., 2002. The 5′ and 3′ extremities of the Satellite tobacco necrosis virus translational enhancer domain contribute differentially to stimulation of translation. RNA 8, 229-236. doi:10.1017.S1355838201018076
Walter, A. E., Turner, D. H., Kim, J., Lyttle, M. H., Muller, P., Mathews, D. H., Zuker, M., 1994. Coaxial stacking of helixes enhances binding of oligoribonucleotides and improves predictions of RNA folding. Proc. Nat. Acad. Sci. USA 91, 9218-9222.
Wang, J., Simon, A. E., 1997. Analysis of the two subgenomic RNA promoters for Turnip crinkle virus in vivo and in vitro. Virology 232, 174-186. doi:10.1006/viro.1997.8550
Wang, S., Guo, L., Allen, E. M., Miller, W. A., 1999. A potential mechanism for selective control of cap-independent translation by a viral RNA sequence in cis and in trans. RNA 5, 728-738.
Weng, Z., Xiong, Z., 1995. A method for accurate determination of terminal sequences of viral genomic RNA. Genome Res. 5, 202-207.
White, K. A., Nagy, P. D., 2004. Advances in the molecular biology of tombusviruses: Gene expression, genome replication, and recombination. Prog. Nucleic Acid Res. Mol. Biol. 78, 187-226.
Wu, B., Vanti, W. B., White, K. A., 2001. An RNA domain within the 5′ untranslated region of the Tomato bushy stunt virus genome modulates viral RNA replication. J. Mol. Biol. 305, 741-756. doi:10.1006/jmbi.2000.4298
Wu, B., White, K. A., 1999. A primary determinant of cap-independent translation is located in the 3′-proximal region of the Tomato bushy stunt virus genome. J. Virol. 73, 8982-8988.
Zhang, G., Slowinski, V., White, K. A., 1999. Subgenomic mRNA regulation by a distal RNA element in a (+)-strand RNA virus. RNA 5, 550-561.
Zuker, M., 2003. Mfold web server for nucleic acid folding and hybridization prediction. Nucleic Acids Res. 31, 3406-3415. doi:10.1093/nar/gkg595

### Example 2

### Determining the Importance of Base-Pairing Interactions Between 5′ and 3′UTRs for MNeSV RNAs In Vitro and In Vivo

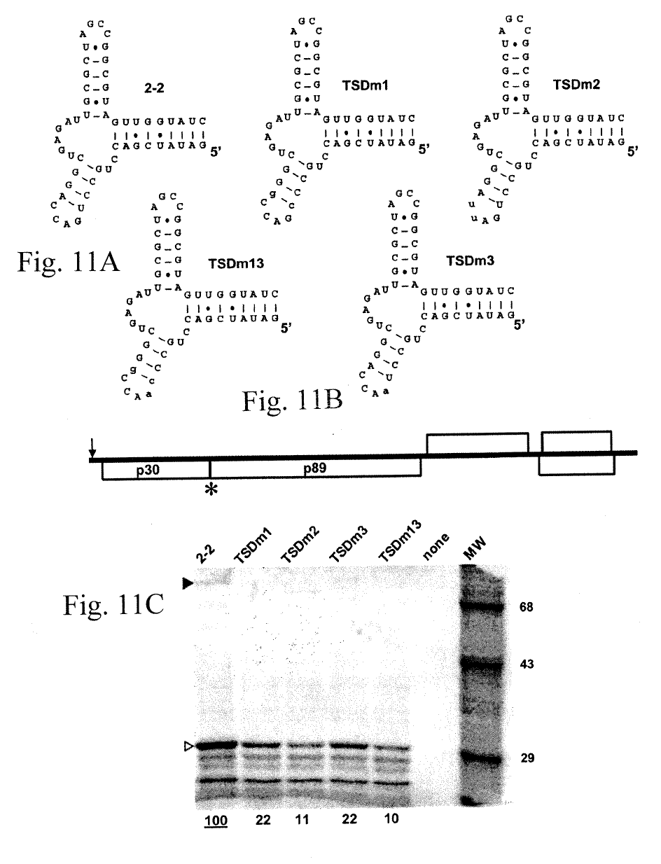

To determine if mutations of one to three nucleotides (nt) in the “kissing” loop of the 5′ UTR of genomic transcripts could decrease translation, four mutant cDNAs (pTSDm1, pTSDm2, pTSDm3, pTSDm13) were produced. The mutations would allow maximum consecutive base-pairing between the T-shaped domain (TSD) and wild type 3′CITE of five to seven base pairs compared to seven base pairs for wild type (WT). Transcripts from pTSDm2 could produce seven base pairs, but two G-U base pairs (2 hydrogen bonds/pair) replace G-C base pairs (3 hydrogen bonds/pair) leading to an overall weaker base-pairing interaction. Equimolar amounts of the full length WT and mutant transcripts were translated in WGE and analyzed by SDS-PAGE (FIG. 11C). As expected, the modified RNAs translated less well than the WT transcript producing 10% (TSDm13), 11% (TSDm2), and 22% (TSDm1 and TSDm3) of WT levels of p30.

This experiment demonstrates that even slight modifications to the KL of the TSD decreased translation indicating that a 5′-3′ base-pairing interaction is required for CITE activity, and that the strength of the base-pairing interaction affects the efficiency of translation.

Further experiments to determine the effect of changing the potential base-pairing interactions between the “kissing” loop of the 3′CITE and sequences in the 5′ UTR of genomic MNeSV RNA and 5′ UTR of sgRNA1 were performed. Transcripts from p2-2-KLC and psg1-KLC contained the same changes in five bases in or near the KL of the 3′CITE that were mutated in psg2-KLC (3′KLC in FIG. 7). These changes would decrease the number of potential consecutive base pairs between the TSD and the 3′CITE from seven to three. When equimolar amounts of uncapped transcripts were translated in WGE, 2-2-KLC transcripts produced 14-15% of the p30 and p89 proteins compared to wild type (2-2) transcript, which is similar to the decreased production of p19 from sg2-KLC transcripts. This experiment demonstrates that the requirement for strong base-pairing interactions between the 5′ and 3′ UTRs of genomic transcripts for strong cap-independent translation is similar to the requirement for a strong base pairing interaction for sgRNA2 transcripts. To determine if the location of the base-pairing sequence in the 5′ UTR of sgRNA1 is important, modifications were made to the 5′ UTR (psg1-KLA), the 3′ UTR (psg1-KLC) or both UTRs (psg1-KLAC ) of the plasmid encoding MNeSV sgRNA1. There are two sequences (“A” and “B”) in the 5′ UTR of MNeSV sgRNA1 that may potentially base pair with the “kissing” loop of the 3′CITE. The sequence of the 5′UTR of transcripts from psg1-KLA is GgaacACAACUCGGCACACAAACGCACACAAC (SEQ ID NO: 23). The four bases near the extreme 5′ end of the 5′UTR were mutated so the wild type 3′CITE kissing loop could only potentially base-pair with two bases of the “A” sequence, but could still base pair with the five nt in the “B” sequence which begins six bases further downstream. Transcripts from psg1-KLAC contained the same mutations in the 5′ UTR “A” sequence and the 3′CITE “KLC” mutation, and this would allow an interaction with five base pairs at the modified “A” sequence, but leave the potential base-pairing at sequence “B” at two base pairs. If the 3′CITE preferentially base pairs with sequence “A”, then translation from sg1-KLA and sg1-KLC should both decrease to similar low levels, and sg1-KLAC should translate similar to sgRNA1. If sequence “B” is the preferred base-pairing site, then sg1-KLA should translate similar to sgRNA1, and sg1-KLC and sg1-KLAC should have greatly reduced translations. Based on experiments with 2-2KLC and sg2-KLC, sg1-KLC was expected to express between 10-20% as much coat protein as sgRNA1. Surprisingly, translation of equimolar amounts of uncapped sgRNA1 and mutant transcripts indicated that the amount of coat protein produced from all mutant transcripts varied from about 50-90% of the wild type level. sg1-KLC translated about half as well as sgRNA1, probably because the 3′CITE of sg1-KLC can potentially base pair with six bases that are located between the “B” sequence and the start codon of the ORF. Thus, all three mutants had one or two possible base-pairing sites. This experiment demonstrates that the ability of the 3′ CITE to efficiently base pair with any sequence in the 5′ UTR of sgRNA1 is more important than the exact location of the sequence in the 5′ UTR, and that the exact sequence of the base-pairing interaction is not important as long as the base changes do not lead to a major change in secondary structure of the 3′ CITE or the 5′ UTR.

Additionally, transcripts from p2-2KLC, pTSDm1, pTSDm2, and pTSDm13 were tested in maize protoplast infection assays. All three transcripts with mutations in the TSD were capable of replication and accumulated both gRNA and sgRNAs indicating that both p30 and p89 were synthesized from gRNA. TSDm1 accumulated gRNA to 21% of the WT level while TSDm2 and TSDm13 accumulated gRNA to less than 4% of the WT level. While it is possible that these mutations disturbed cis-acting replication signals, it is more likely that the decrease in viral replication was a direct effect of producing lower levels of the viral replication proteins as in similar experiments with TBSV (Fabian and White 2004). Since sgRNA1 and sgRNA2 produced during replication of these mutants were the same as WT sgRNAs, synthesis of CP, p19, and p21 would occur as efficiently as translation of sgRNAs in a WT infection. 2-2KLC transcripts were unable to replicate suggesting that the KLC mutation made the 3′CITE nonfunctional in vivo since replication and synthesis of sgRNAs requires synthesis of p30 and p89. These experiments demonstrate that small mutations that weaken the base-pairing interaction between the KLs in the 5′ UTR and 3′CITE decreased the ability to synthesize both p30 and p89 in vivo, and that greater changes (KLC mutation) in the 5′-3′ base-pairing interaction essentially inactivated the 3′CITE activity in vivo.

### Example 3

### Decreasing the Distance Between the 5′ and 3′ UTRs Increases Translation in Wheat Germ Extract

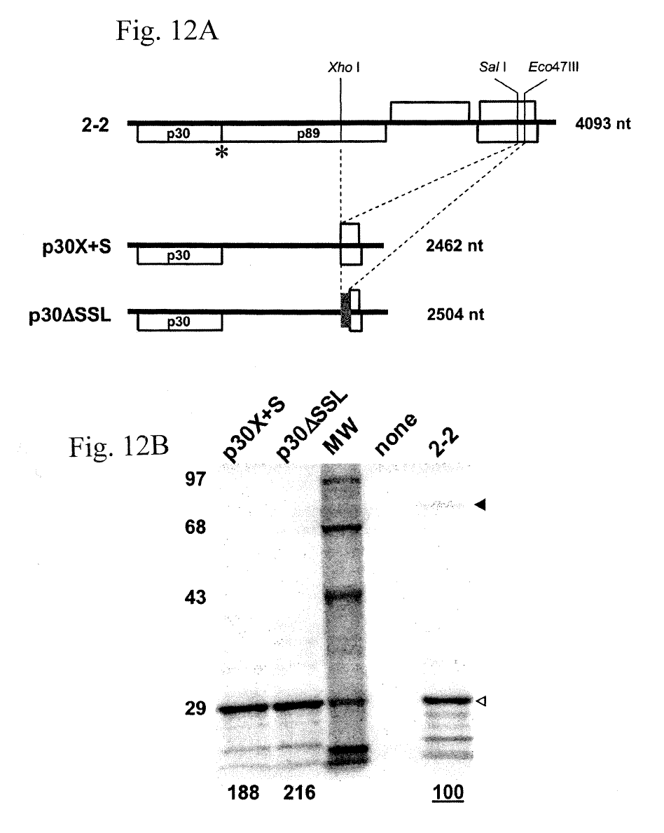

Previous work indicated that the translation efficiency of MNeSV RNAs was sgRNA2>sgRNA1>gRNA which is indirectly proportional to the length of the viral RNAs. The ability of the 5′ and 3′ UTRs of these RNAs to interact may depend on the context of the sequence in the 5′ UTR, the distance between the 5′ and 3′ UTRs, or both. To determine if the distance is an important factor, deletion mutants of p2-2 were constructed that removed about 40% of the DNA between the p30 ORF stop codon and the 3′ UTR leaving the p30 ORF as the only intact coding region (FIG. 12A). Equimolar amounts of full length and shortened transcripts were translated in WGE and analyzed by SDS-PAGE. The shorter RNAs synthesized about twice as much p30 as the full length RNA (FIG. 12B). This experiment demonstrates that moving the 5′ UTR and 3′UTR closer together increases translation in vitro when using the same context for the KL in the 5′ UTR.

### Example 4

### Expression of a Heterologous Protein from a 3′CITE Vector in Wheat Germ Extract

The coding region of plasmid pNsg1 is replaced by the restriction site NaeI. This produces a plasmid vector (psg1EXPRESS) in which the T7 RNA polymerase promoter is followed by the 5′ UTR of MNeSV sgRNA1, a unique NaeI site, and the 3′ UTR of MNeSV followed by a unique SmaI site. The complete coding sequence for firefly luciferase is amplified using appropriate primers using the polymerase chain reaction, and the ends of the amplified DNA are treated with a nuclease to remove any overhanging bases. The luciferase DNA fragment is inserted into psg1EXPRESS at the NaeI site by ligation with T4 DNA ligase. The DNA is transformed into competent, Escherichia coli, and a colony containing the recombinant plasmid with the luciferase coding sequence in the correct orientation is isolated. Recombinant plasmid DNA (psg1LUC) from this clone is linearized with SmaI and used to synthesize uncapped RNA using T7 RNA polymerase. The transcript is added to a complete wheat germ extract translation system to produce large amounts of luciferase. An assay for luciferase activity shows that a high level of active protein is expressed from this uncapped RNA.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of producing a protein, polypeptide or peptide translated from an uncapped eukaryotic messenger ribonucleic acid (mRNA), comprising the steps of:
a) providing a DNA molecule comprising sequences encoding
i) an RNA polymerase promoter;
ii) a 5′ untranslated region (UTR) of a cap-independent translation enhancer (CITE) from a tombusvirid or a tombusvirid satellite virus, wherein said tombusvirid naturally infects monocots;
iii) an open reading frame (ORF) encoding a protein; and
iv) a 3′ UTR of a CITE from a tombusvirid or a tombusvirid satellite virus;
b) allowing transcription of an uncapped mRNA molecule; and
c) allowing translation of said uncapped mRNA molecule to produce said protein, polypeptide or peptide.

2. The method of claim 1, wherein
said DNA molecule further comprises a restriction site immediately downstream of said 3′ UTR;
said method is carried out in an in vitro translation system; and
said step of allowing transcription comprises contacting said DNA molecule with an RNA polymerase under conditions where said RNA polymerase binds to said RNA polymerase promoter and causes transcription of said sequences encoding said 5′ UTR of a CITE from a tombusvirid or a tombusvirid satellite virus, said ORF, and said 3′ UTR of a CITE from a tombusvirid or a tombusvirid satellite virus, into said uncapped mRNA molecule.

3. The method of claim 2, wherein said in vitro translation system is a wheat germ extract translation system.

4. The method of claim 1, wherein said DNA molecule further comprises sequences encoding a ribozyme and an RNA polymerase terminator sequence immediately downstream of said 3′ UTR, and said method takes place within a eukaryotic cell.

5. The method of claim 4, wherein said eukaryotic cell is a monocot.

6. The method of claim 1, wherein said tombusvirid is Maize necrotic streak virus.

7. The method of claim 1, wherein said protein, polypeptide or peptide is a heterologous protein, polypeptide or peptide.

8. The method of claim 7, wherein said heterologous protein is selected from the group consisting of enzymes, fluorescent proteins, fusion proteins, structural proteins, transport proteins, regulatory proteins, and storage proteins.

9. A DNA molecule comprising sequences encoding
an RNA polymerase promoter;
a 5′ UTR of a CITE from a tombusvirid or a tombusvirid satellite virus, wherein said tombusvirid naturally infects monocots;
an open reading frame (ORF) encoding a protein, polypeptide or peptide; and
a 3′ UTR of a CITE from a tombusvirid or a tombusvirid satellite virus, wherein said tombusvirid naturally infects monocots.

10. The DNA molecule of claim 9, further comprising a restriction site immediately downstream of said 3′ UTR.

11. The DNA molecule of claim 9, further comprising sequences encoding a ribozyme and a RNA polymerase terminator sequence immediately downstream of said 3′ UTR.

12. The DNA molecule of claim 9, wherein said protein, polypeptide or peptide is a heterologous protein, polypeptide or peptide.

13. The DNA molecule of claim 12, wherein said heterologous protein is selected from the group consisting of enzymes, fluorescent proteins, fusion proteins, structural proteins, transport proteins, regulatory proteins, and storage proteins.

14. An isolated and substantially purified RNA molecule comprising the nucleic acid sequence UGAUGUGAGGAACGUGGACUGUGAUGUGGUGGUGCGGUACCAUGGC UGGUCACCAUGGUAAUGCGUAGGGCAACACAGUUCAUUAAGACUCACUGAUG AUGGCACUAGGCACGGUUCACCCCCAUCCUUCGGGAGGGCUAUAGGGGGUGA CCGGGUUACACCACCGGAAGACCGGAACAUUGCCUUUGGGCAGCCC (SEQ ID NO: 1).

15. An isolated and substantially purified RNA molecule comprising the nucleic acid sequence AGAUAUCGACCUGCCUGACCAGGCUGAGAUUGCGCUAGCCGGCGUA GUUGGUAUCUCUCGCGCAAGCGGGUUUGAAGGUGCGGCCUACCUUAGGGGGG UAAAUUGUAACUUCGCACAAAGGC (SEQ ID NO: 2).

16. An isolated and substantially purified RNA molecule comprising the nucleic acid sequence GACCAACAACUCGGCACACAAACGCACACAAC (SEQ ID NO: 3).

17. An isolated and substantially purified RNA molecule comprising the nucleic acid sequence GAACAAGACCAGUUCAUGGAUGCAGAAUACGAGCAAGUCAGU AGGCC (SEQ ID NO: 4).

18. An RNA molecule, comprising:
a 5′ UTR of a CITE from a tombusvirid or a tombusvirid satellite virus, wherein said tombusvirid naturally infects monocots;
a 3′ UTR of a CITE from a tombusvirid or a tombusvirid satellite virus, wherein said tombusvirid naturally infects monocots; and
a heterologous set of nucleotides coding for a protein of interest.

19. The RNA molecule of claim 18, wherein said 5′ UTR is represented by SEQ ID NO: 2, SEQ ID NO: 3, or SEQ ID NO: 4, and the 3′ UTR is represented by SEQ ID NO: 1.
